(19) United States
(12) Patent Application Publication
(10) Pub. No.: US 2010/0312046 A1
(43) Pub. Date: Dec. 9, 2010

(54) ASSEMBLY AND METHOD FOR AUTOMATICALLY CONTROLLING PRESSURE FOR A GASTRIC BAND

(75) Inventors: Lilip Lau, Los Altos, CA (US); Yi Yang, San Francisco, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: CAVU MEDICAL, INC., Los Altos, CA (US)

(21) Appl. No.: 12/832,308

(22) Filed: Jul. 8, 2010

Related U.S. Application Data
(63) Continuation of application No. 12/322,163, filed on Jan. 29, 2009.

(51) Int. Cl. A61F 2/04

(52) U.S. Cl. 600/37

(57) ABSTRACT

An elastic bladder is provided that is in constant fluid communication with the expandable balloon portion of a gastric band in order to automatically and continuously adjust the gastric band. The fluid pressure between the bladder and the balloon portion of the gastric band automatically and continuously adjusts so that there is no lasting pressure differential between the bladder and the expandable balloon. As the level of restriction imparted by the gastric band on the stomach of the patient changes, fluid from the bladder automatically and substantially instantaneously flows to or from the expandable balloon portion of the gastric band thereby maintaining neutral fluid pressure equilibrium between the bladder and the balloon and automatically adjusting the band to the correct level of restriction to keep the patient in the optimum zone for weight loss.

### CROSS-REFERENCES TO RELATED APPLICATIONS

This application claims priority from U.S. application Ser. No. 12/322,163, filed Jan. 29, 2009, incorporated by reference in its entirety.

### BACKGROUND

### Field of the Invention

The present invention relates to the field of treating obesity using a laproscopic adjustable gastric band or lap band. As the patient loses weight, the gastric band is adjusted to accommodate for changes in weight.

Laparoscopic adjustable gastric banding was rapidly embraced as a procedure for treating morbid obesity after its introduction in Europe and in the United States. Compared to Roux-en-Y gastric bypass, the existing gold standard bariatric surgery procedure, it was attractive because it was safer, with one-tenth the peri-operative mortality, less morbid, easier and faster for surgeons to learn and perform, required a shorter hospital stay and resulted in a faster post-operative recovery. In addition, the device and the degree of restriction that it provided could be adjusted to suit the patient at different points in time. If necessary, the device could be removed surgically. The procedure involves no permanent alteration of the patient's anatomy. In addition, the patients are free of many of the side effects that accompany the malabsorption of the gastric bypass such as hair loss, anemia and the need to take supplemental vitamins. These attributes were attractive both to the health care providers and to the patients.

However, laparoscopic adjustable gastric banding has some drawbacks. Weight loss and co-morbidity resolution do not occur as rapidly as with gastric bypass surgery, with most reported results trailing in weight loss at one, two, three and possibly four years. In addition, there is considerably more variability from patient to patient in the amount of weight that they lose. More recent data has suggested that over time, the difference diminishes because gastric bypass results show an early peak in weight loss followed by subsequent decline. At five years there does not appear to be a statistical difference in weight loss between bypass and gastric banding (Surgery for Obesity and Related Diseases 1, pp. 310-316, 2005).

One current method for treating morbid obesity includes the application of a gastric band around a portion of the stomach to compress the stomach and create a narrowing or stoma that is less than the normal interior diameter of the stomach. The stoma restricts the amount of food intake by creating a pouch above the stoma. Even small amounts of food collecting in the pouch makes the patient feel full. The patient consequently stops eating, resulting in weight loss. It is important to maintain the right level of restriction imparted by the band in order for the patient to feel full and thereby to have continuous and uniform weight loss. Prior art gastric bands include a balloon-like section that is expandable and deflatable by injection or removal of fluid from the balloon through a remote injection site such as a port near the surface of the skin. The balloon expandable section is used to adjust the correct level of restriction imparted by the band both intraoperatively and postoperatively. Currently, patients must return to the doctor as many as four to ten times per year for several years in order to have fluid injected into or removed from the balloon in order to maintain the correct level of restriction imparted by the band.

It was first reported by Forsell and colleagues in 1993 (“Gastric banding for morbid obesity: initial experience with a new adjustable band”; Obes. Surg. 1993; 3:369-374) that individuals with adjustable gastric bands experienced plateaus in their weight loss during the time between scheduled adjustments. A typical weight loss curve is shown in FIG. 1A.

In 2008, Rauth, et al. (“Intra-band pressure measurements describe a pattern of weight loss for patients with adjustable gastric bands”; J. Am. Coll. Surg. 2008; 206; 5:926-932) reported that “patients commonly attribute this pattern of weight loss to a ‘loosening’ of their band, stating that the band provides progressively less restriction during meals and less satiety between them.” Rauth, et al. described a clinical study that uses a manometer to measure the intra-band pressure of the adjustable gastric bands in vivo during routine postoperative adjustments. The group recorded significant intra-band pressure drops between adjustments and proposed that such loss of band pressure, which could not be explained solely by band volume loss, not intra-band volume, led to plateaus in weight loss and results in patients' observations that the band becomes looser with time as shown in FIG. 1B.

Rauth, et al. suggested that the loss of band pressure was due to remodeling of the tissue that is occupied by the inner circumference of the band. They hypothesized that during the first 60 days after band insertion, there remains considerable perigastric fat and some residual tissue edema; the volume of the encircled stomach is greatest. As weight is lost and edema resolves, the volume of stomach contained within the band decreases, resulting in less contact pressure between the tissue and the band which in turn results in a decrease in intra-band pressure per unit intra-band volume.

In order to be efficacious and safe, frequent follow-up visits to the physician, most of which involve band adjustments, are necessary. Some have described this as the Achilles heel of gastric banding. In fact, studies have shown a correlation between weight loss and the number of band adjustments or office visits that a patient undergoes (Shen). The band adjustments are usually performed in the setting of a physician's office. In these procedures saline is added or removed from the band in order to adjust it to the right tightness or restriction. Many factors are considered in making this adjustment. The goal is to try and tune the band to a “sweet spot” or “green zone.” In this zone the patients are able to adhere to proper eating patterns and lose one to two pounds per week.

Gastric Band Adjustment To Optimize Weight Loss

| | GREEN ZONE | |
|---|---|---|
| Add Fluid | Fluid Level Optimum | Remove Fluid |
| Patient is hungry | Patient not hungry, | Patient makes poor food |
| between meals, eating | good weight loss, | choices, experiences |
| large portions, and | food portion control, | regurgitation, discomfort |
| not losing weight | patient satisfaction | while eating, poor weight |
| | | loss, night coughing |
| Not enough fluid | Right amount of | Too much fluid in the |
| in the band | fluid in the band | band |

Current gastric band adjustment protocols vary from physician to physician and also depend on the feedback provided by the patient. Most physicians currently leave the band empty for the first six weeks or so after the surgery in order for the band to heal in place. The healing involves a foreign body response in which inflammation and fibrosis lead to encapsulation of the band. Typically, this process subsides over time in the absence of further stimulation. After this initial settling in period adjustments to the band begin. Adjustments typically can be categorized into two phases: the initial careful incremental adjustment into the green zone followed by the subsequent maintenance of the green zone by tuning the band to either tighten or loosen it to achieve the desired restriction. Conventional adjustment practice involves adding or removing prescribed increments of saline (e.g., 0.5 cc) to the band and then double checking the level of restriction by having the patient sit up and drink water or barium under fluoroscopic imaging. In the initial phase increments of saline are added up to or starting from a target volume (e.g., 4 cc). As can be expected, there is considerable patient to patient variability as to the intra-band volume and number of adjustments that initially bring them into the proper adjustment of the green zone. Typically, two to five adjustments are needed to attain the green zone initially.

Once the patients attain the green zone, subsequent adjustments are performed to keep them there. In the first year after band implantation there may be two to five additional adjustments to maintain the green zone. Most often this involves adding saline or tightening the band on a monthly or so basis. This is performed if the patient falls out of the green zone. More commonly this is in response to inadequate rate of weight loss which often coincides with patients reporting that their bands have loosened or are loose. The exact mechanism behind the loosening is not clear, but several factors have been suggested. Some leakage of saline may occur out of the band over time. Air is often trapped in the band initially which may dissolve or dissipate over time. Epi-gastric fat is often encircled by the band and with time this may go away. The stoma itself and the fibrous cap around the band may remodel over time. What is clear though is that the addition of sometimes small amounts of saline into the band will bring back the feeling of restriction to the patients.

Occasionally, gastric bands need to be loosened as well. If the band is too tight or tightened too quickly the patient may feel excessive restriction. The patient may have a difficult time eating with frequent episodes of vomiting. Also, certain foods may get stuck. Ironically, this may lead to weight gain as patient learns to cheat the restriction provided by the band by drinking milkshakes and other liquid foods. Another more serious drawback of excessive tightening is that the band may erode through the stomach wall if it is left in that state. Swelling or edema can cause the band to become too tight. Patients report that bands may be tighter feeling in the morning and looser later in the day. Female patients often report feeling increased tightness around the time of their menstrual cycles. Usually, removing fluid from the band can relieve this tightness.

Band adjustments are still performed beyond the first year but less frequently. Patients may come in on a quarterly basis, especially during the second and third year.

Despite the recognition of the criticality of band adjustments, patient compliance remains an issue. Some patients may not come in for adjustments when required. Many patients live considerable distances from the surgeon who implanted their band. The need for frequent adjustments can be very demanding on these patients in terms of the time away from work and cost of travel. In the extreme case, many patients opt to have their bands implanted out of the country because of cheaper costs. After their procedure they cannot afford to travel out of the country for frequent band adjustments. some patients move and subsequently have difficulty finding a surgeon to perform their adjustments. Even within the U.S. some surgeons will not adjust the bands of patients that were not implanted by them for fear of potential liability.

Further, there is the direct cost of adjustments. Typically, even when the surgery is reimbursed by insurance, the adjustments are not, or even when they are, they are inadequately reimbursed. The patient may not be able to afford the out-of-pocket fees for adjustments which often can be several hundred dollars per adjustment. Finally, there are complex psychological motivational obstacles that prevent them coming in for the necessary adjustments. For example, some patients have a fear of the syringe needle that is used to inject saline into the band.

The inconvenience of adjustments is not limited to the patients. Surgeons generally do not like the need for frequent adjustments. Historically, they are not accustomed to the intensive long term care of their patients. Many do not have the existing infrastructure within their practices to manage the post-procedural aftercare of the patients. This consists of having the staff to perform adjustments, providing counseling, psychologists, nutritionists, nurses, etc. In addition, as surgeons implant more and more bands, the pool of patients that will need adjustments grows. Consequently they may end up spending less time operating and a considerable amount of time performing adjustments.

Without adjustments patients experience interrupted or cessation of weight loss and even weight regain. If the bands are too loose the patients eating habits may regress. Even if they are aware of this it often can take time for them to schedule and receive a proper adjustment. If the bands are too tight and not adjusted they not only are uncomfortable, but patients may adopt bad eating habits, such as drinking milkshakes. In the extreme case they can experience erosion of their bands into the stomach or esophagus which would necessitate band removal.

Even if the patients are compliant and can overcome the barriers to attending follow-up visits adjustments can be problematic. Locating the subcutaneous fill port can be difficult. Sometimes the port will move or flip over. In these cases fluoroscopy or even surgical revision are needed. Repeated needle punctures can lead to infection. Actual adjustment protocols can differ from surgeon to surgeon. Different bands have different pressure-volume characteristics which can lead to even greater inconsistency. The adjustment protocols were derived from trial and error and not any physiological basis. Even after a patient is properly adjusted changes may occur very shortly afterward, within days to weeks, that create a need for another adjustment.

It is clear that the less the need for adjustments the better the gastric banding therapy will be. Weight loss results will be more uniform from patient to patient and less dependent on follow up. The amount of weight lost and the rate at which it is lost will also be better because of less interrupted weight loss. Co-morbidity resolution will also improve accordingly. Less need for band adjustments would also result in cost and time savings to both the patients and healthcare providers. Reducing the variability in outcomes, increasing the rate and amount of weight loss and reducing the need for follow-up visit adjustments combined with the inherent present advantages of gastric banding would create a bariatric surgery potentially that would offer the best of gastric bypass and banding. Many more patients may opt for this procedure than previously would have chosen bypass or banding.

Current band adjustments are highly variable if measured in terms of volume, which is the current adjustment metric. Rauth, et al.'s group reported substantial variability in intra-band volume that can produce similar intra-band pressure as shown in FIG. 1C. Patient #39's intra-band pressure reached 730 mmHg at the intra-band volume of 2 ml while patient #43's intra-band pressure reached similar level (758 mmHg) at the intra-band volume of 4 ml, a difference of 2 ml which is 50% of the entire intra-band volume capacity (see FIG. 1C).

Also, other published papers suggest that a narrow range of intra-band pressure based on a more physiological approach might achieve good weight loss and prevent esophageal problems in the long term. Lechner and colleagues (“In vivo band manometry: a new access to band adjustment”; Obes. Surg.; 2005; 15:1432-1436) reportedly adjusted a cohort of twenty-five patients to a basic pressure of 20 mmHg at the first band filling. None of the patients returned to the clinic due to obstruction. In a continuation of this work, Fried reported that when patients that had previously lost less than 40% EWL with banding, they were adjusted to 20-30 mmHg intra-band pressure using manometry, resulting in significant weight loss at 12 weeks. Both Lechner, et al. and Fried, et al. suggested that the gastric band adjustment based on pressure might be more physiologic, accurate and reliable. Furthermore, Gregersen in his book titled “Biomechanics of the Gastrointestinal Tract” stated that the normal resting pressure “in the lower esophageal sphincter generally lies between 10 and 40 mmHg above atmospheric pressure.” Thus, it would seem reasonable to have band-tissue contact pressure near this range.

One drawback common among the prior devices that use some type of device to fill and replenish fluid in the balloon portion of the band is that their pressure-volume compliance curves are relatively steep. In other words, for each incremental fill volume (i.e., 0.5 ml), there is a correspondingly large increase in intra-band pressure. Published prior art pressure volume curves are disclosed in Ceelen, Wim, M. D., et al., Surgical Treatment of Severe Obesity With a Low-Pressure Adjustable Gastric Band: Experimental Data and Clinical Results in 625 Patients, Annals of Surgery, January 2003, pp. 10-16; Fried, Martin, M. D., The current science of gastric banding: an overview of pressure—volume theory in band adjustments, Surgery for Obesity and Related Diseases, 2008, pp. S14-S21; Rauth, Thomas P., M. D., et al., Intraband Pressure Measurements Describe a Pattern of Weight Loss for Patients with Adjustable Gastric Bands, Journal of American College of Surgeons, 2008, pp. 926-932; Lechner, Wolfgang, M. D., et al., In Vivo Band Manometry: a New Access to Band Adjustment, Obesity Surgery, 2005, pp. 1432-1436; Forsell, Peter, et al., A Gastric Band with Adjustable Inner Diameter for Obesity Surgery: Preliminary Studies, Obesity Surgery, 1993, pp. 303-306 which are incorporated herein by reference thereto.

What has been required in the art is a device that automatically adjusts the fluid level in the gastric band to maintain it and the entire system at or near the intra-band and/or contact pressure at which the band was last adjusted to. The present invention provides a device for passively equalizing pressure in a closed fluid system that automatically and continuously equalizes the pressure in the system in order to maintain the proper restriction to keep the patient in the so-called “green zone” in a prescribed pressure range.

### SUMMARY OF THE INVENTION

The present invention relates generally to the treatment of obesity using a gastric band or lap band to wrap around a portion of the stomach thereby producing a stoma which limits the amount of food intake of the patient. The gastric band has an adjustable fluid balloon which can be expanded or deflated in order to provide the right level of restriction to the stomach of the patient. In one embodiment of the invention, an inflatable bladder is provided that is in constant fluid communication with the expandable balloon-portion of the gastric band. The fluid volume in the bladder and the balloon automatically and continuously adjusts back and forth so that there is no lasting pressure differential between the expandable balloon and the bladder, and in so doing, the pressure in the balloon is maintained even if there are changes in fluid volume in the balloon in response to changes in loading from the surrounding tissue or if there is some leakage of the fluid from the balloon.

In one embodiment, an assembly for passively equalizing pressure in a closed fluid transfer system includes a bladder having an internal volume for receiving a fluid and an expandable balloon section having an internal volume for receiving a fluid. The bladder is configured so that the fluid in the bladder is under pressure and it takes on or expels fluid as governed by its pressure-volume relationship or compliance. The fluid within the bladder is under pressure because the bladder itself is elastic, thereby applying pressure on the fluid within. The expandable balloon is associated with the inner portion of the gastric band surrounding the stoma. As the level of forces on or around the gastric band change, fluid from the bladder automatically and substantially instantaneously flows to or from the expandable balloon thereby equalizing fluid pressure between the bladder and balloon and automatically adjusting the band to the correct level of restriction to keep the patient in the green zone. In this embodiment, the neutral fluid pressure between the bladder and the balloon is governed by the pressure-volume relationship, or compliance of the bladder, which in turn alters the pressure-volume relationship of the entire system. The balloon/band has a compliance that can be measured. The bladder also has a compliance that can be measured. The combination of the bladder and the balloon/band has a compliance that is different than that of the balloon or the bladder alone with a lower pressure at certain volume ranges. The compliance is the slope of the pressure-volume curve and that slope can change as a function of fill volume. Over certain operating volume ranges, the slope of the combined system will be less than that of the band/balloon alone.

The compliance of the bladder is such that it can keep the pressure of the band within a desired range even if: (1) the band loses up to 5 cc of fluid; (2) the band gains up to 5 cc of fluid volume; (3) the stoma encircled by the band increases in diameter; and (4) the stoma encircled by the band decreases in diameter.

In another embodiment, an assembly for passively equalizing pressure in a closed fluid system includes a bladder having an internal volume for receiving a fluid. The bladder is enclosed in a rigid housing to protect the bladder from external forces such as body tissue in the area of the implanted bladder. The bladder is in fluid communication with an expandable balloon associated with the gastric band. As the loading on the gastric band changes, fluid from the bladder automatically and substantially instantaneously flows to or from the expandable balloon thereby maintaining neutral fluid pressure between the bladder and balloon and automatically adjusting the band to the correct level of restriction to keep the patient in the green zone.

In another embodiment, an assembly for passively equalizing pressure in a closed fluid system includes a bladder having an internal volume for receiving a fluid. The bladder is enclosed in a rigid housing to protect the bladder from external forces such as body tissue in the area of the implanted bladder. The bladder is in fluid communication with an expandable balloon associated with the gastric band. As the level of restriction imparted by the gastric band changes, fluid from the bladder automatically and substantially instantaneously flows to or from the expandable balloon thereby maintaining neutral fluid pressure between the bladder and balloon and automatically adjusting the band to the correct level of restriction to keep the patient in the green zone. In this embodiment, the bladder is in fluid communication with a port that is internally implanted in the patient, and near the surface of the skin. In order to replenish any fluid in the bladder, fluid can be injected through the port which will then flow into the bladder and replenish any fluids in the system.

In another embodiment, the tubing extending from the balloon portion of the gastric band to a fill port contains an expandable lumen with the desired compliance characteristics. In this embodiment, the tubing can have multiple lumens with an elastic or deformable wall separating the different lumens. As with the other embodiments, as the loading on the gastric band changes, the fluid from the expandable tubing (bladder) automatically and substantially instantaneously flows to or from the expandable balloon thereby maintaining neutral fluid pressure between the expandable tubing/bladder and the expandable balloon and automatically adjusts the band to a level of restriction to keep the patient in the green zone.

In another embodiment, a rigid housing contains a bladder that is elastically compressible (e.g., the bladder containing air, foam, sponge materials, micro-bubbles, or similar compressible materials). Fluid within the housing surrounds the bladder and as changes on the loading of the gastric band occur, fluid from the housing surrounding the compressible bladder will automatically and substantially instantaneously flow to or from the expandable balloon in the gastric band. In this embodiment, the bladder can be initially pressurized with air, which is compressible, and the fluid surrounding it and contained within the housing will act to compress the bladder, thereby generating pressure within the fluid in the housing.

In another embodiment, the expandable balloon portion of the gastric band is in fluid communication with a device that has a fluid pressure that is higher than the fluid pressure in the expandable balloon. As the loading on the gastric band changes, fluid from the device automatically and substantially instantaneously flows to or from the expandable balloon thereby maintaining neutral fluid pressure between the device and the balloon and automatically adjusting the band to the correct level of restriction to maintain the patient in the green zone. In this embodiment, the device has a compliance that is lower than the compliance of the expandable balloon of the gastric band.

### BRIEF DESCRIPTION OF THE DRAWINGS

FIG. 1 is a schematic of a prior art gastric band system depicting a balloon portion of the gastric band and fill port.

FIG. 1A depicts a typical prior art weight loss curve.

FIG. 1B depicts a typical prior art weight loss curve.

FIG. 1C depicts a graph depicting the variability in intra-band volume as it relates to intra-band pressure.

FIG. 1D depicts a graph of experimental data showing intra-band pressure dropping when a mandrel diameter encircling the band decreases.

FIG. 1E depicts a graph of intra-band pressure and volume curves resulting from experimental data.

FIG. 1F depicts a graph resulting from experimental data in which a bladder was incorporated between a gastric band a fluid infusion port.

FIG. 1G depicts a graph resulting from experimental data in which a bladder was able to change the intra-band pressure/volume characteristics of a gastric band.

FIG. 2 is a schematic view of a bladder assembly having elastomeric bands to add elasticity to the system.

FIG. 3 is a longitudinal sectional view of the bladder assembly of FIG. 2.

FIG. 3A depicts a graph of experimental data resulting from experiments on the bladder disclosed in FIGS. 2 and 3.

FIG. 4 depicts a schematic view of a bladder assembly encased in a housing.

FIG. 5A depicts a longitudinal cross-sectional view of one embodiment of the bladder assembly of FIG. 4.

FIG. 5B depicts a longitudinal cross-sectional view of an alternative embodiment of the bladder assembly of FIG. 4.

FIG. 5C depicts a graph of experimental data relating to the embodiment of the bladder shown in FIGS. 4, 5A and 5B.

FIG. 6 depicts a longitudinal cross-sectional view of a bladder assembly having multiple bladders encased in a housing.

FIG. 7 depicts a longitudinal schematic view of a bladder assembly having multiple bladders encased in a housing.

FIG. 8 depicts a longitudinal schematic view of multiple bladder assemblies aligned serially.

FIG. 8A depicts a graph of experimental data relating to the embodiment of the bladder shown in FIG. 8.

FIG. 9 depicts a schematic view of a bladder assembly housed in a fill port assembly.

FIG. 10 depicts a top cavity of the injection portion bladder assembly of FIG. 9.

FIG. 11 depicts a schematic view of a bottom cavity of the injection port bladder assembly of FIG. 9 with the bladder substantially unfilled.

FIG. 12 depicts an enlarged view of the bottom cavity of the injection port bladder assembly of FIG. 9 without a bladder.

FIG. 13 depicts an exploded schematic view depicting the top cavity and the bottom cavity of the injection portion bladder assembly of FIG. 9 with the bladder being substantially filled.

FIG. 14 depicts a schematic view of a bellows-type bladder assembly encased within a housing.

FIG. 15 depicts a longitudinal schematic view of a multi-compliant bladder assembly housed within a solid housing.

FIG. 16 depicts a multi-level pressure compliance curve associated with the multi-compliant bladder assembly of FIG. 15.

FIG. 17A depicts a schematic view of a gastric band assembly with a bladder assembly in form of tubing.

FIG. 17B depicts a cross-sectional view taken along lines 17B-17B showing a coaxial bladder and tubing assembly.

FIG. 17 C depicts a cross-sectional view taken along lines 17C-17C showing a bladder and tubing assembly having an elastic septum.

FIG. 18 depicts linearly increasing and decreasing compliance curves.

FIG. 19 depicts a flat or substantially constant pressure compliance curve.

FIG. 20 depicts a multi-staged substantially constant pressure curves.

FIG. 21 depicts multi-staged linearly increasing compliance curves.

FIG. 22A depicts an exponentially increasing pressure compliance curve.

FIG. 22B depicts a logarithmic increasing compliance curve.

FIGS. 23 and 24 depict a schematic view of a gastric band assembly with a bladder system and a sensor to monitor pressure or other parameters.

FIG. 25 depicts a schematic view of a bladder system incorporated into a venous access catheter assembly.

FIG. 26 depicts a schematic view of a gastric band assembly having an elastic balloon.

FIG. 27A depicts a plan view of a bladder having a longitudinal fold.

FIGS. 27B-27C depicts a cross-sectional view of the longitudinal fold of FIG. 27A; FIG. 27B shows the folded configuration and FIG. 27C shows the unfolded configuration.

### DETAILED DESCRIPTION OF THE PREFERRED EMBODIMENTS

At present, typical prior art gastric banding systems include a gastric band having an expandable balloon section and tubing extending from the balloon to a port. The port is implanted near the surface of the skin so that fluid can be injected into the port with a syringe in order to add fluid to the balloon section thereby adjusting the level of restriction. One such typical gastric banding system is disclosed in U.S. Pat. No. 6,511,490, which is incorporated by reference herein.

The present invention embodiments generally include one or more bladders in constant fluid communication with the expandable balloon section of the gastric band to automatically and continuously minimize the drops or rises in pressure from the properly adjusted level and in doing so the proper level of restriction provided by the band in order to keep the patient in the green zone. The bladder(s) is a passive system that does not require motors, drive pumps, or valves, nor does it require a feedback sensor to measure pressure or the level of restriction.

Several experiments, as reported below, were conducted to determine the relationship between: (1) changes in diameter of the stoma versus intra-band pressure (i.e., pressure in the balloon section); and (2) changes in fluid volume in the balloon section versus the corresponding changes in intra-band pressure (i.e., balloon pressure). The intra-band pressure is defined as the pressure generated by both the contact pressure between the stomach tissue and the band, and the balloon inflation pressure which is the pressure it takes to inflate the balloon portion of the gastric band. There may be other factors that influence the intra-band pressure, such as intra-abdominal pressure. However, the main factors contributing to the intra-band pressure are the contact pressure between the stomach tissue and the band, and the pressure it takes to inflate the balloon.

### Experiment No. 1

An in vitro model was constructed to show that a bladder could transfer fluid to or from an expandable balloon on a gastric band in response to controlled changes in the size of the stoma encircled by the balloon. To simulate the changes in volume of the encircled stomach tissue/stoma, an aluminum mandrel with varying diameter from 20 mm to 8 mm was fabricated. Each diameter segment was about 2.5 mm in length along the mandrel. At the end of the 8 mm diameter segment, the mandrel diameter increased to 2.5 mm, large enough to be held with a pair of soft jaw clamps that were then secured to a stand at a height such that the subject mandrel diameter segment was just above another soft jaw clamp positioned lower on the same stand. A Realize band (Ref #RLZB22 made by Ethicon Endo-Surgery, Inc., a Johnson & Johnson company) was slid over the subject mandrel segment such that the band encircled the mandrel. Part of the band where the silicone tubing was connected laid on top of the lower clamp. The reference inlet of a manometer was also attached to the lower soft jaw clamp. A 10 cc syringe was attached to a 3-way stopcock. A 22 gauge Huber tip needle was connected to the stopcock port directly across from the syringe. The pressure reading inlet of the manometer was attached to the side port of the 3-way stopcock and was held in place with a vice. Finally, the Huber tip needle was used to puncture the access port of the Realize band system.

The Realize band was then placed around the 20 mm diameter segment of the mandrel and the band was supported by the lower soft clamp. A vacuum was drawn with the 10 cc syringe to remove as much air inside the balloon of the band as possible. Water was slowly injected into the access port of the reservoir until the intra-band pressure reached about 30 mmHg. The valve of the three-way stopcock to the syringe port was closed and the intra-band pressure was recorded after the system had reached a steady state. The Realize band was moved from the 20 mm diameter segment to the 18 mm diameter segment of the mandrel and the mandrel was lowered so that the 18 mm diameter segment was at the same height as the 20 mm diameter segment had been. The intra-band pressure was recorded after the system had reached a steady state. The steps above were repeated for both mandrel diameter segments of 16 mm and 14 mm.

By varying the mandrel diameter that was encircled by the Realize band, the change in stomach tissue volume/stoma diameter was simulated in an in vitro model. The experiment showed that intra-band pressure dropped significantly when the mandrel diameter that was encircled by the band decreased, as shown in FIG. 10. Just as Rauth, et al. had hypothesized, the intra-band pressure drop could be related to the decreasing volume of stomach contained within the band.

In addition to Rauth, et al.'s explanation of patients feeling the loosening of the band in between adjustments, Dixon, et al. documented some leakage of saline out of the band over time. Also, others suggested that trapped air inside the band may dissolve or dissipate over time. Both saline leakage and air dissolution would result in a decrease in intra-band volume and hence a decrease in intra-band pressure.

### Experiment No. 2

The Realize band was placed over and encircled the 20 mm diameter segment of the mandrel. Part of the band was supported by the lower soft clamp. A vacuum was drawn using the 10 cc syringe to remove as much air as possible from inside the expandable balloon section of the band. The balloon section of the band was next inflated with water in 0.5 ml increments for a total of 9 ml. The intra-band pressure was recorded per each increment increase. The balloon section of the band was next deflated in 0.5 ml decrements and the intra-band pressure was recorded per each decrement and the intra-band pressure was recorded per each decrement.

To demonstrate that intra-band volume change can affect intra-band pressure, the in vitro model described above was used to characterize the volume-pressure relationship of the Realize band.

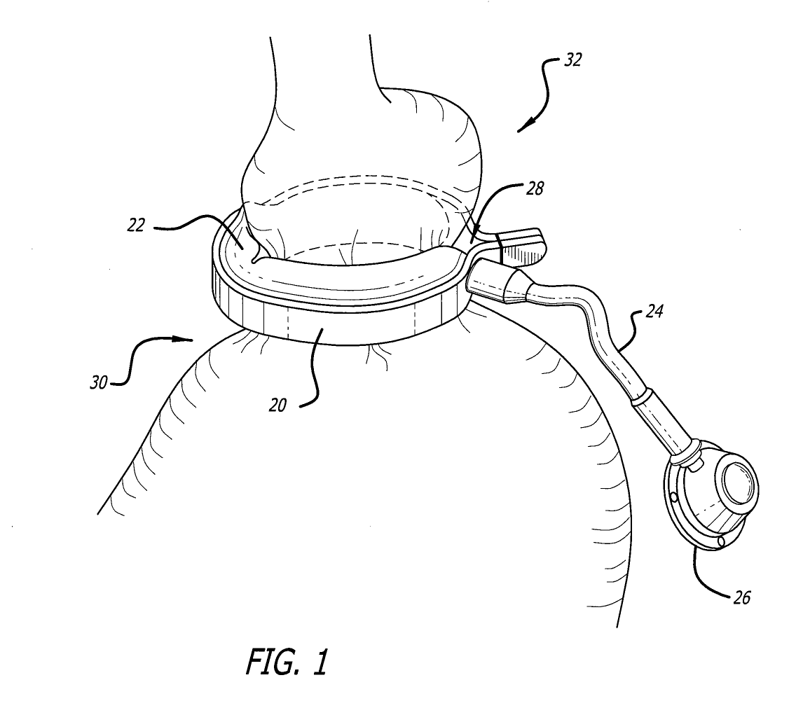

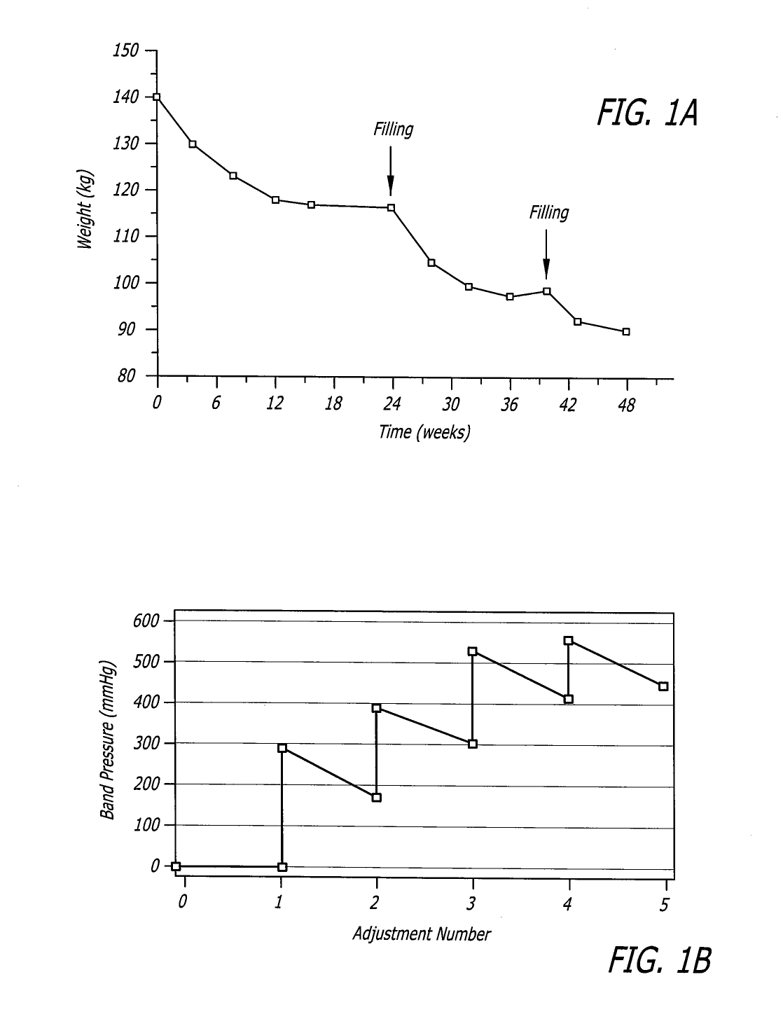

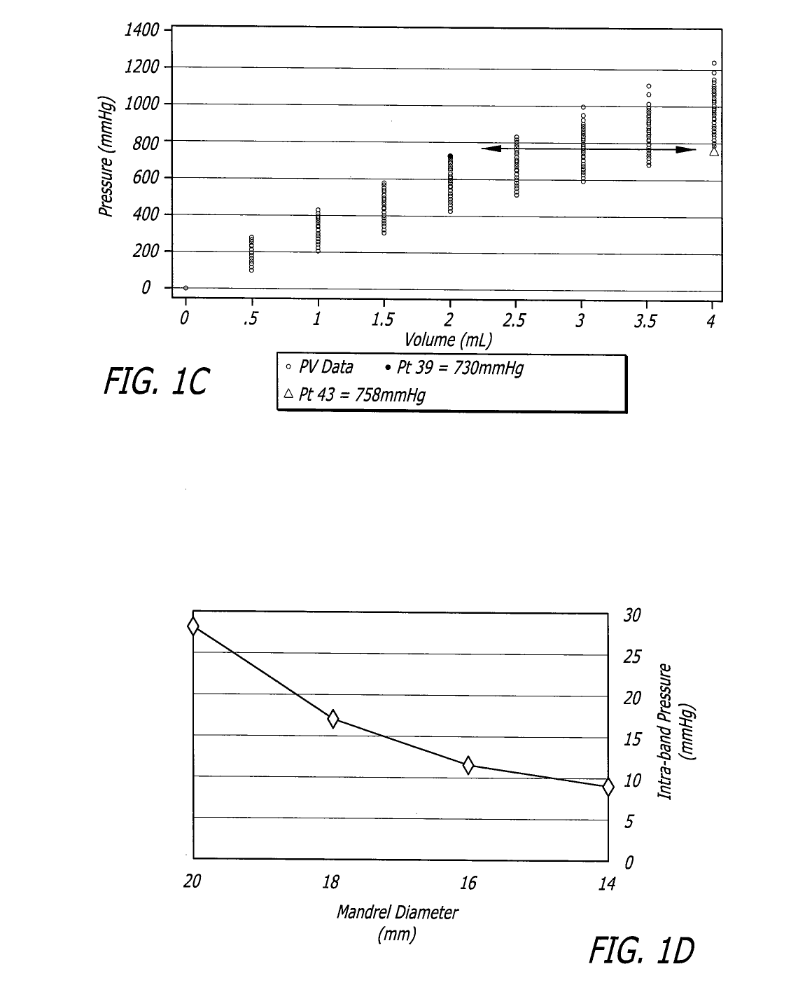

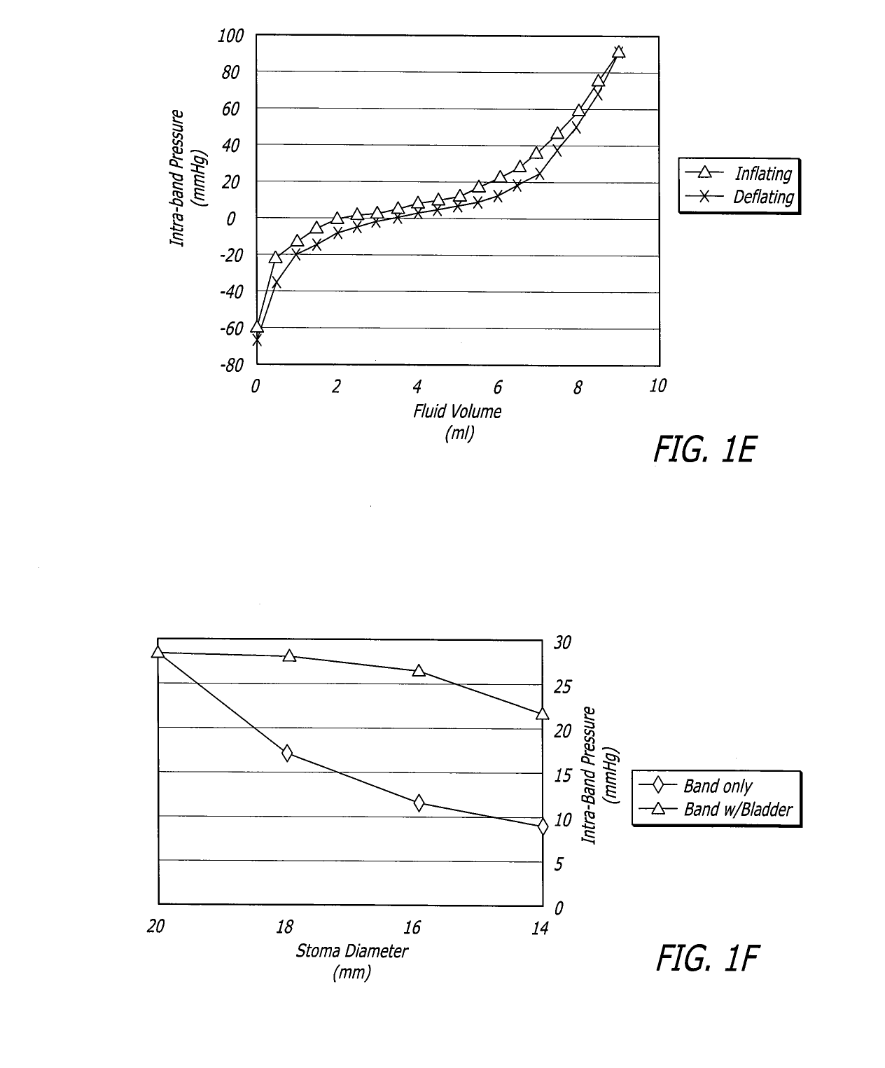

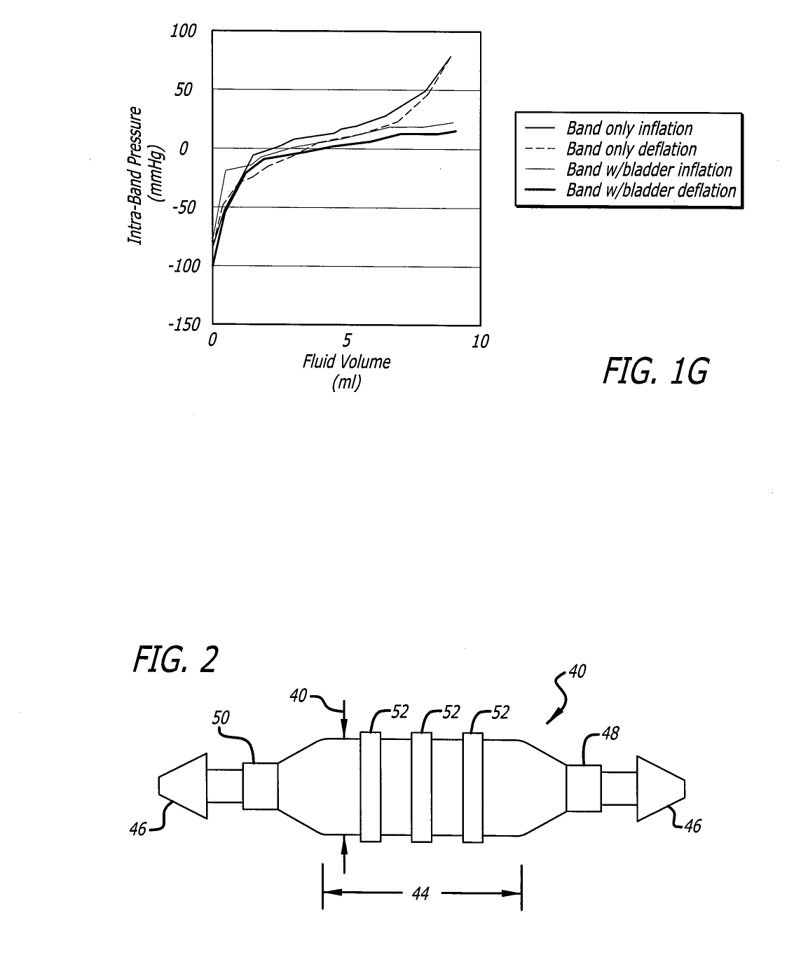

This experiment showed that the intra-band pressure increased with an increase in volume and decreased with a decrease in volume of the expandable balloon. Furthermore, the data showed that the rate of pressure change for a given change in fluid volume increased significantly as the intra-band volume reached its full capacity, which has important clinical implications discussed in detail below. The intra-band pressure and volume curves are shown in FIG. 1E.

The two experiments demonstrated in vitro that both change in stomach tissue volume and change in intra-band fluid volume could affect the intra-band pressure. However, the exact mechanism behind the feeling of band loosening in between adjustments may not be clear. What is clear though is that the addition of small amounts of fluid into the band as is done during the majority of the band adjustments can bring back the feeling of restriction and satiety to the patients.

### Experiment No. 3

In this experiment, a bladder or fluid reservoir was incorporated between the Realize gastric band and a standard fluid infusion port. The bladder was filled with a fluid and was in fluid communication with the infusion port and the balloon portion of the gastric band. The bladder had a lower compliance than the balloon portion of the gastric band, therefore the bladder will fill the gastric band as the inner diameter of the band is reduced. The in vitro experiments described in Experiment 2 were repeated and measurements were taken of the intra-band pressure both with and without the bladder in the system. The data is shown in FIG. 1F.

The data shows that the bladder maintained the intra-band pressure over a wide range of encircled tissue volume change as it was simulated by varying (reducing) the mandrel diameter. As the mandrel diameter decreased from 20 mm to 14 mm, the intra-band pressure dropped only 6.5 mmHg (23%) in the system with the bladder versus a drop of 19 mmHg (68%) in the system without the bladder.

### Experiment No. 4

In this experiment, it was demonstrated that the intra-band pressure could be maintained when the bladder was connected in between the Realize gastric band and the fluid infusion port. In this experiment, a vacuum was drawn to remove as much air from inside the balloon portion of the gastric band as possible. Thereafter, the balloon portion of the gastric band was inflated with water in 0.5 ml increments for a total of 9 ml. The intra-band pressure was recorded at each increment. Thereafter, the balloon portion of the gastric band was deflated in 0.5 ml decrements and the intra-band pressure was recorded at each decrement. As demonstrated by the data, the bladder was able to change the intra-band pressure/volume characteristics of the gastric band. As can be seen in FIG. 1G, the slope of the curve of the gastric band with the bladder is much flatter than that of the slope of the curve of the gastric band without the bladder in the system, especially in the 6 to 9 ml volume range. The distance is even more pronounced when the intra-band pressure exceeded 10 mm Hg. The bladder also acted as a regulator so that the intra-band pressure would not exceed a predetermined limit.

Based on the experiments above, a novel pressure bladder could be added to existing gastric bands. Such a bladder would maintain the intra-band pressure over a wider range of intra-band fluid volume change or encircled tissue volume or tissue-band loading change. By preventing the intra-band pressure from dropping or rising appreciably, patients would be maintained in the “green zone” longer, thus reducing the number of adjustments necessary or even potentially eliminating adjustments altogether.

This novel bladder is a passive system having a specific predetermined pressure-volume curve inherent to the system. Based on physiological and clinical observations, the bladder of the present invention works in the pressure range between 10-50 mmHg for certain types of commercially available gastric bands, but for some gastric or lap bands, the pressure range could be between 40 mmHg and 150 mmHg. The pressure-volume compliance curve of the bladder could have a substantially constant pressure over a wide range of volume changes, or multi-plateau pressure settings, or linear etc., as will be shown.

As shown in FIG. 1, a typical prior art gastric band assembly 20 includes an expandable or inflatable balloon section 22 that is connected to tubing 24 in fluid communication with a port 26. The band 20 forms a restriction or stoma 28 so that the stomach 30 has pouch 32 formed above the band. The bladder of the present invention is incorporated into the gastric band assembly 20.

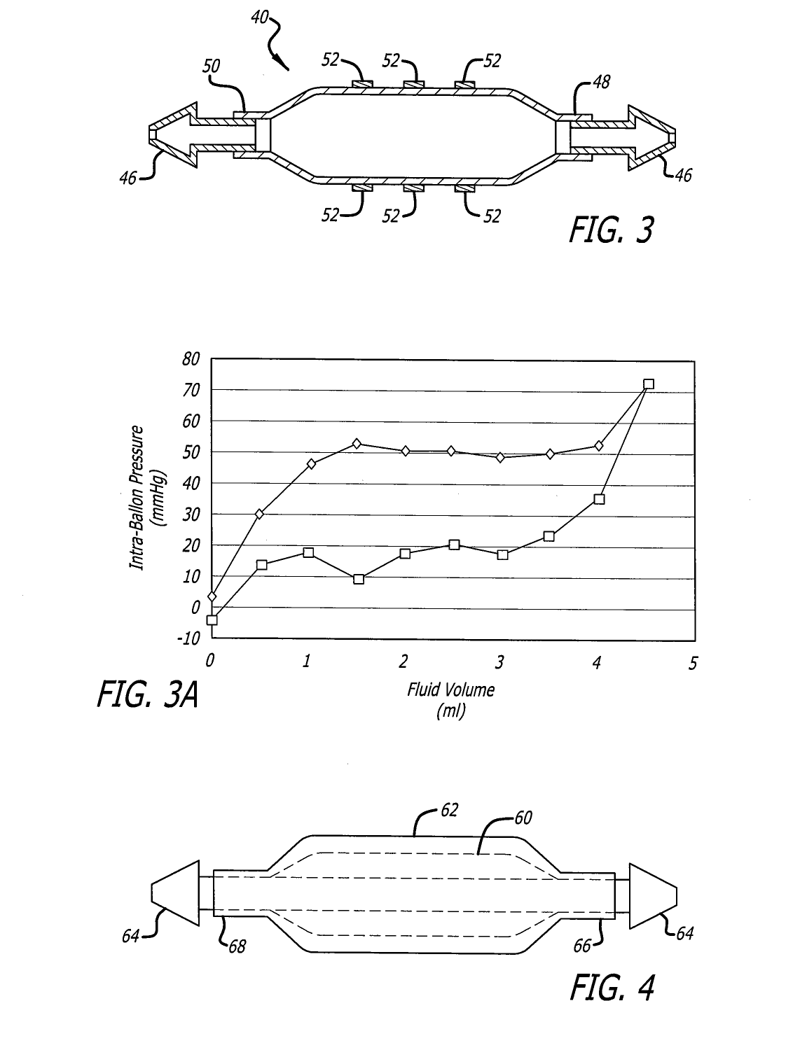

In one embodiment of the present invention, as shown in FIGS. 2 and 3, a bladder 40 has an outside diameter 42 of no greater than about 15 mm and a length 44 of about 14.0 cm. Importantly, the bladder 40 can take on many different shapes and dimensions. For example, the bladder can have any shape (elongated, tubular, cylindrical, toroidal, annular, and the like), and it can be configured to receive from 0 to 14 ml of fluid. The bladder is formed from an elastic material such as polyethelene, silicone rubber, urethane, ePTFE, nylon, stainless steel, titanium, nitinol, cobalt chromium, platinum, and similar materials approved for implanting an in humans. A barbed fitting 46 is attached to the bladder's infusion lumen 48 and discharge lumen 50. Three elastomeric bands 50 are positioned on the outer surface of the bladder with a spacing of about 7 mm between the bands. The bands are made out of synthetic polyisoprene (HT-360 by Apex Medical Technologies) and are highly elastic. In this embodiment, the bladder is substantially inelastic. The bands have an inside diameter of about 5.7 mm, width of about 4.57 mm, and a wall thickness of 0.127 to 0.1651 mm. In this embodiment, the bladder 40 can be incorporated into any typical gastric banding assembly such as that shown in FIG. 1. The bladder 40 would be connected to tubing 24 shown in FIG. 1 by inserting the luer fittings 46 in the tubing so that the bladder 40 was in line with the tubing 24 situated between the port 26 and the balloon 22. The infusion lumen 48 of the bladder 40 is inserted into the tubing 24 toward the port 26, while the discharge lumen 50 of the bladder 40 is inserted into the tubing 24 in the direction of the balloon 22. The bladder 40 can be inserted into any commercially available gastric banding assembly having at least an expandable balloon portion, while it is not necessary to include the port as described.

The bladder of the present invention can be characterized as an expandable waterproof container with a defined pressure-volume relationship that, when hooked up to a balloon portion of a gastric band, alters the pressure volume relationship of the balloon system, making its compliance curve flatter. The bladder of the present invention can be elastic, pseudo-elastic, or exhibit other characteristics, but it is biased to return to a resting low volume state from a stretched or filled state. The bladder can be an expandable balloon or bellows, made of plastic, metal, or rubber (or a combination of these materials). It is impermeable to saline, contrast media, and similar materials, although it may leak slightly over time. The bladder is made of any biocompatible material and is MRI compatible. The bladder is durable, reliable and fatigue resistant. If the bladder ruptures, the system is still functional and can still be adjusted by adding and removing saline or other fluid. The present invention bladder can be located anywhere in the system, even within the balloon portion of the gastric band. The bladder can be located in the connecting tubing between the balloon portion of the gastric band and the fill port, within the fill port, or as a separate component of the system. The bladder may or may not have a protective shell or housing surrounding the bladder. Such a shell or housing provides protection to the bladder and also acts as a limit to the expansion or distension of the bladder. When the bladder is filled with fluid, any further filling above a certain volume will result in a significant rise in pressure. The surgeon will be able to feel this pressure through the syringe used to fill the bladder. This acts as a tactile set point for the surgeon. For example, the surgeon may fill the band until this significant rise in pressure is felt, and then remove some fluid, perhaps 1 cc, so that the bladder not only has room to contract, but also to expand if the balloon portion of the gastric band feels an increased squeeze or pressure.

The embodiment of the bladder 40 disclosed in FIGS. 2 and 3 was tested to establish a intra-balloon pressure versus fluid volume chart as seen in FIG. 3A. The test results showed that there were two pressure plateaus where the intra-bladder pressure was maintained over a range of intra-bladder fluid volume. During bladder 40 inflation (the upper curve), a pressure plateau around 50 mmHg was formed when fluid volume increased from 1.5 ml to 4 ml, a range of 2.5 ml. During bladder deflation (the lower curve), a second pressure plateau around 20 mmHg was formed when fluid volume decreased from 3.5 ml to 1 ml, a range of 2.5 ml. This phenomenon was not expected since the polyethylene bladder alone (without the bands 52) did not exhibit similar pressure/volume characteristics. It is the combination of the bands 52 elasticity and the unfolding/folding of the non-elastic bladder that created this pressure/volume curve. Consequently, different plateaus are achieved with different band elasticity and bladder folding geometries.

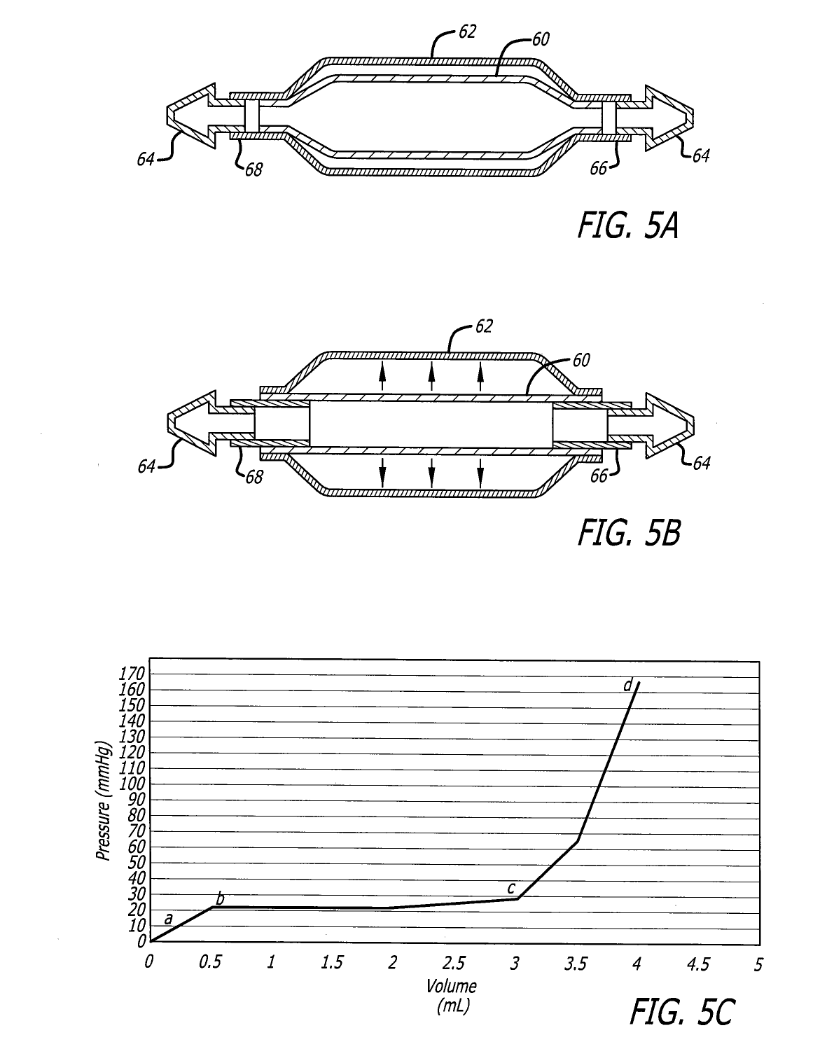

In another embodiment, as shown in FIGS. 4 and 5A and 5B, a bladder 60 having an outside diameter not to exceed 15 mm, is encased in a hard plastic housing 62. Barbed fittings 64 are attached to the infusion lumen 66 and discharge lumen 68 of the housing 62. In this embodiment, the bladder is formed of an elastomeric material which could be in the form of a tube. The bladder 60 could be made out of any number of elastomers from which specific and desired pressure-volume compliance curves can be controlled by the dimensions of the elastomeric tubing, and the type of polymer used in the tubing material. Importantly, bladder 60 is housed within housing 62 so that as the bladder is inflated with a fluid through the infusion lumen 66, the bladder 60 will expand until it contacts the inner walls of housing 62. The housing 62 isolates the bladder from surrounding tissue and limits the total volume that the bladder can expand. Further, the housing 62 will alter the pressure-volume compliance curve of the bladder as seen FIG. 5C. As with the other embodiments disclosed herein, bladder 60 and housing 62 can be incorporated into any gastric banding system such as the one shown in FIG. 1. Further, the housing is fluid tight and acts as a fail-safe mechanism in the event the bladder 60 leaks, and the balloon 22 associated with the gastric band 20 will still function as if the bladder 60 was not present in the system. In other words, fluid can still be injected through port 26 (FIG. 1) and tubing 24, and through the bladder 60 which is encased in the hard shell housing 62 so that fluid will still reach balloon 22. As shown in FIG. 5C, before bladder 60 is inflated, pressure rises as the volume increases (graph segment a-b). As the bladder is inflated, the pressure is held constant (at about 20 mmHg) even though the volume inside the bladder 60 increases from about 0.6 ml to about 3.0 ml (graph segment b-c). Once the bladder 60 is completely full and pressing against the inside wall of housing 62, the pressure rises dramatically as the volume increases (graph segment c-d).

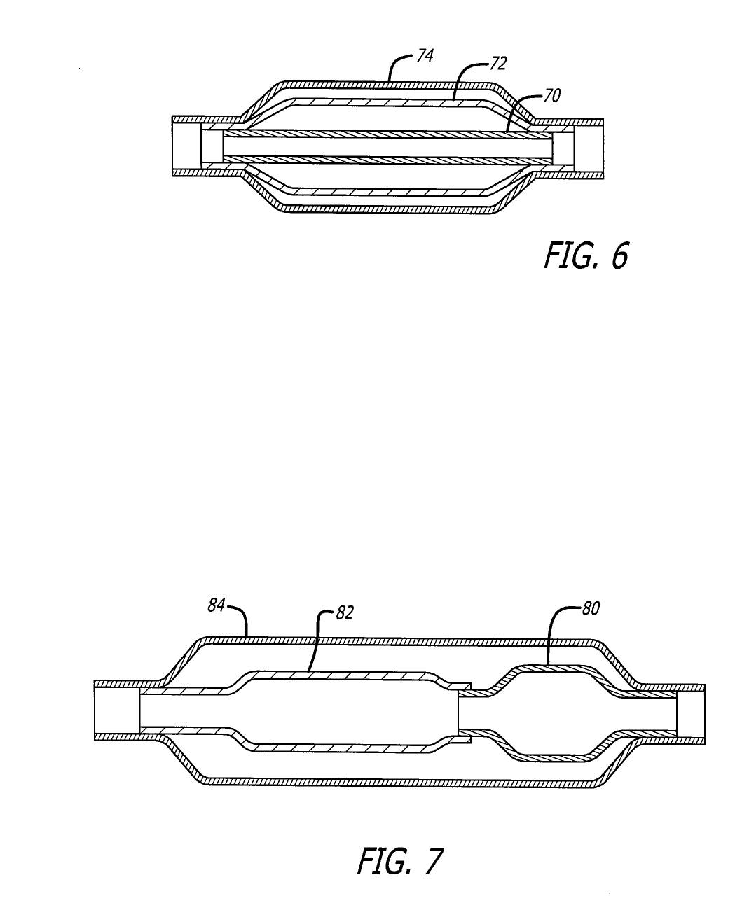

In an alternative embodiment, as shown in FIG. 6, more than one bladder can be used in the system in order to create multiple pressure-volume characteristics. For example, in the FIG. 6 embodiment, a first bladder 70 and a second bladder 72 both are housed in a hard plastic housing 74. The barbed fittings from previous embodiments are not shown for clarity. In this embodiment, the compliance of first bladder 70 is substantially higher than the compliance of the second bladder. As fluid is injected into the first bladder 70, it will easily expand until it comes into contact with the second bladder. Since the second bladder has less elasticity than the first bladder, it will begin to expand well after the first bladder is expanded. As the volume continues to increase, the second bladder also will expand until both the first bladder 70 and the second bladder 72 can no longer expand because the second bladder contacts housing 74. In this embodiment, the second bladder 72 will have a higher constant pressure plateau than the first bladder 70.

In a similar embodiment to that shown in FIG. 6, two bladders can be connected in series within a single housing to effect two different constant pressure plateaus. As shown in FIG. 7, first bladder 80 has a higher elasticity than second bladder 82. Both bladders are encased in housing 74 and, as with FIG. 6, the luer fittings have been omitted for clarity. As fluid is added to the system, first bladder 80 is designed to fully expand into contact with housing 84 before the second bladder 82 begins to expand. After first bladder 80 is fully expanded, second bladder 82 will expand as more fluid is injected into the system until second bladder 82 contacts housing 84. The pressure/volume curves for this embodiment are expected to be similar to that shown in Table 4. Both embodiments shown in FIGS. 6 and 7 can be incorporated into an existing gastric banding system such as the one shown in FIG. 1.

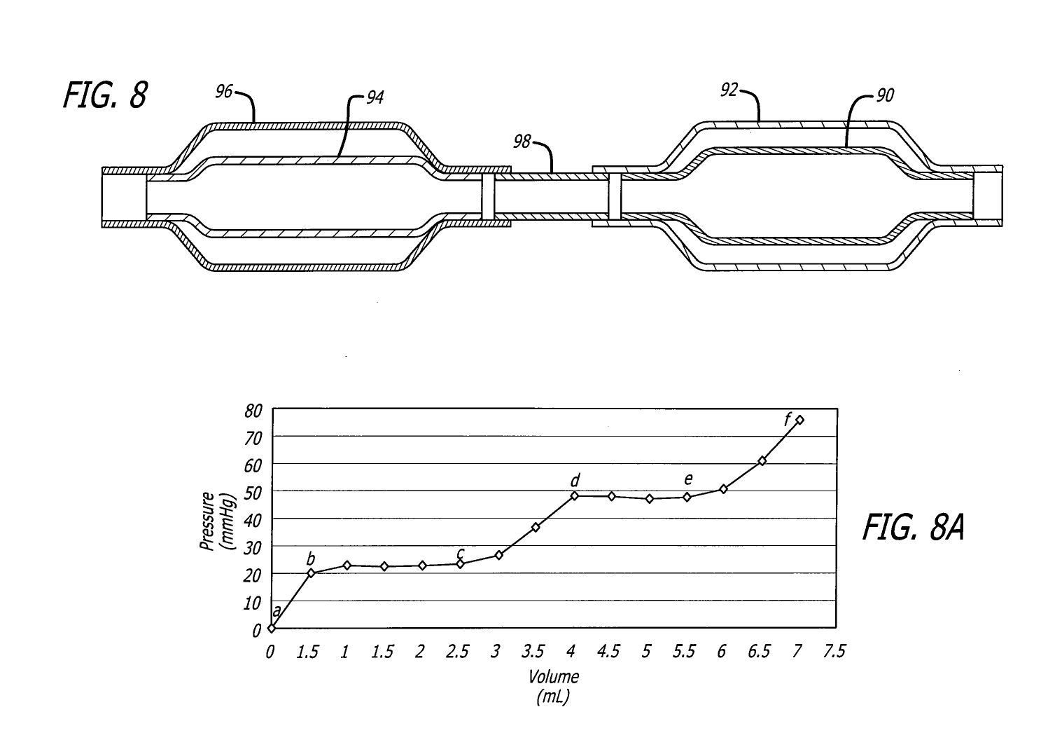

In another embodiment, as shown in FIG. 8, a first and second bladder are arranged serially or in line in separate housings. In this embodiment, first bladder 90 is encased within hard plastic first housing 92 and is in serial fluid communication with second bladder 94 which is encased in hard plastic second housing 96. In this embodiment, first bladder 90 is more elastic than is second bladder 94, so that as the fluid is injected into first bladder 90 it will expand until it contacts the inner surface of first housing 92, before second bladder 94 begins to expand. A tubing 98 is used to connect the housings. As with the other embodiments, the luer fittings have been omitted for clarity. In this embodiment, second bladder 94 has a higher constant pressure plateau than the first bladder 90. Before first bladder 90 begins to inflate, the pressure is held constant (about 20 mmHg) even though the volume increases (from 0.5 to 2.5 ml) as can be seen in FIG. 8A. in the graph segment b-c. Once first bladder 90 fills the entire cavity of the first housing 92, the pressure rises as volume increases, as shown in graph segment c-d. As the volume continues to increase, second bladder 94 will start to inflate and the pressure is once again constant, albeit at a higher pressure level (about 50 mmHg in graph segment d-e) than the constant pressure level exhibited by the filling of first bladder 90. As the second bladder 94 fills the entire cavity of second housing 96, the pressure again rises as the volume increases as shown in graph segment e-f. This embodiment also can be incorporated into any gastric banding system, such as that shown in FIG. 1.

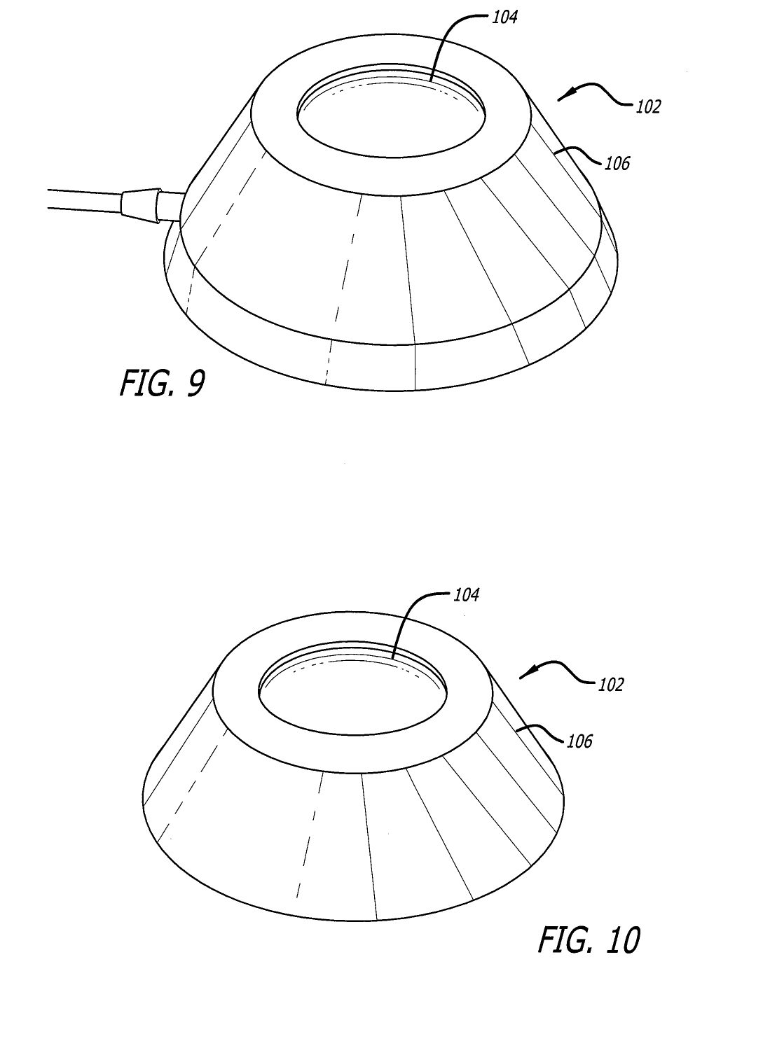

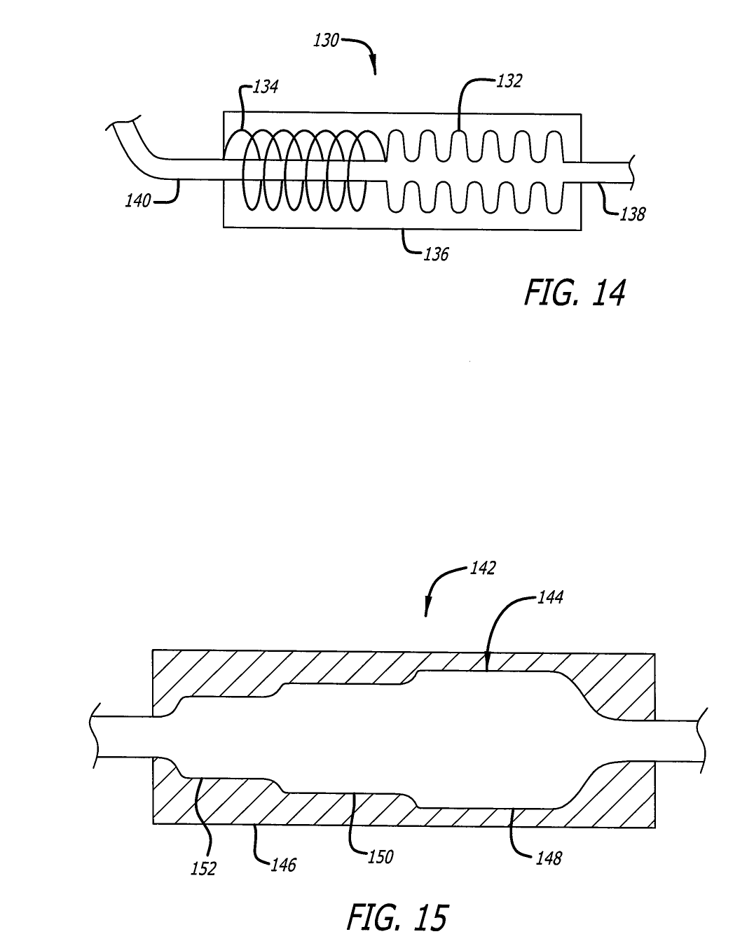

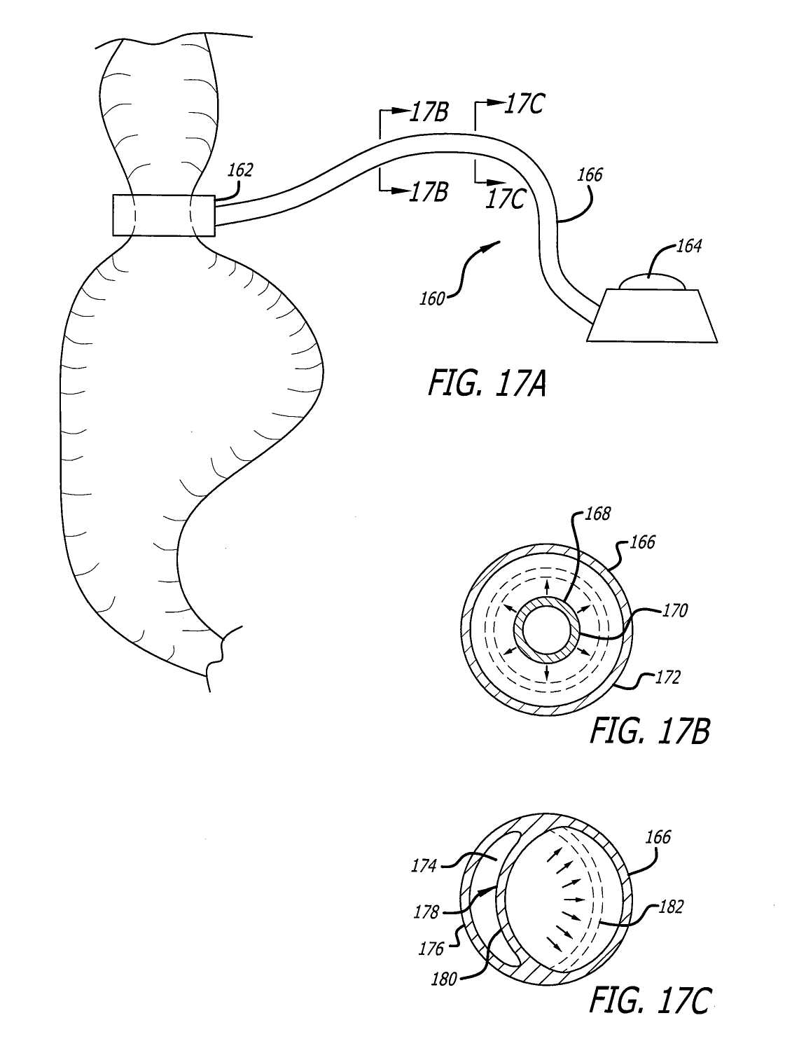

In another embodiment, as shown in FIGS. 9-13, an injection port bladder assembly 100 houses an expandable bladder and is designed to be mounted toward the surface of the skin so that fluid can be injected with a needle to replenish fluids in the system. The injection port bladder assembly 100 is comprised of a housing 102 made of a hard shell plastic, such as polysulfone or titanium, or a combination of both. Housing 102 can be molded or machined. The housing includes a septum 104 which is a self-sealing silicone rubber seal positioned in the top cavity 106 of housing 102. Fluid is injected into the housing by puncturing septum 104 with a needle, and after fluid is injected into the housing, the needle is removed and the septum 104 automatically seals to prevent leakage. The top cavity 106 mates with bottom cavity 108 and the two halves of the housing 102 are sealed together in a known manner. The top and bottom cavity 108 contains expandable bladder 110 in the form of an annular, circular or toroidal configuration. In this embodiment, the bladder 110 can have other configurations and still reside in cavity 108. For example, the bladder could be formed of coaxial tubing similar to that shown in FIGS. 17A and 17B, it could have a septum (FIGS. 17A and 17C), it could have a bellows configuration (FIG. 14), or it could be donut, disk or irregular-shaped, as long as the bladder fits in cavity 108. More broadly, bladder 110 can have any shape that allows it to flex or deform elastically thereby imparting pressure on the fluid within the system consistent with the compliance curves disclosed herein.

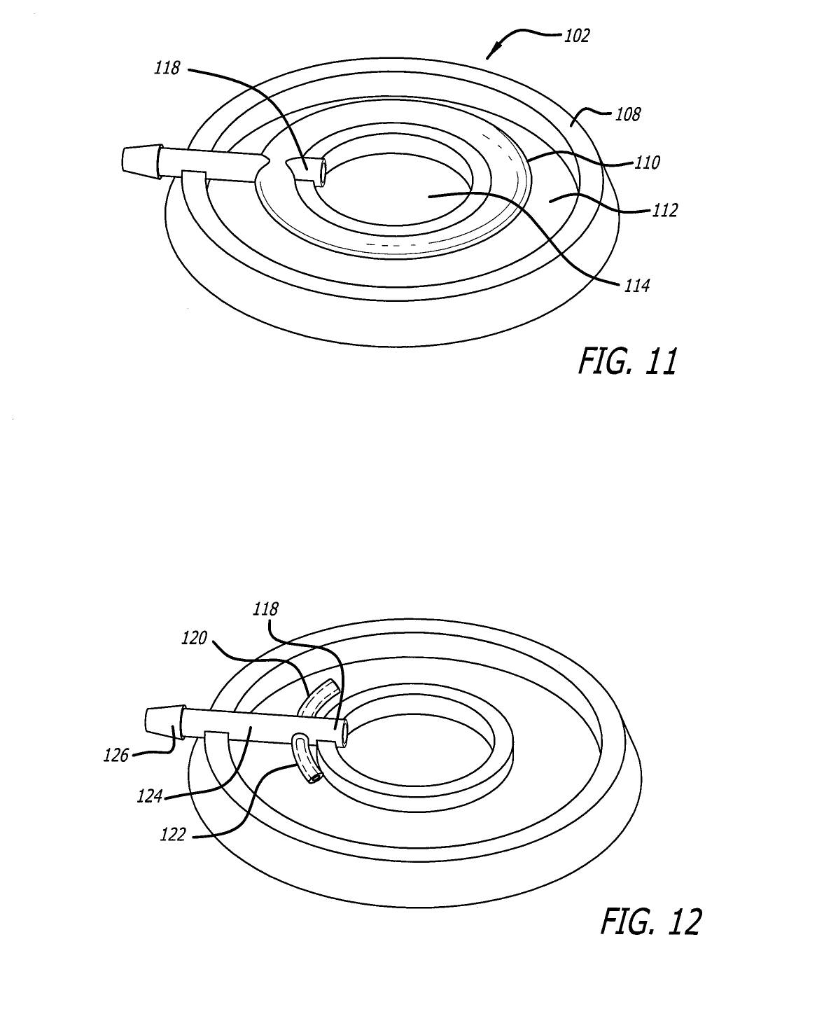

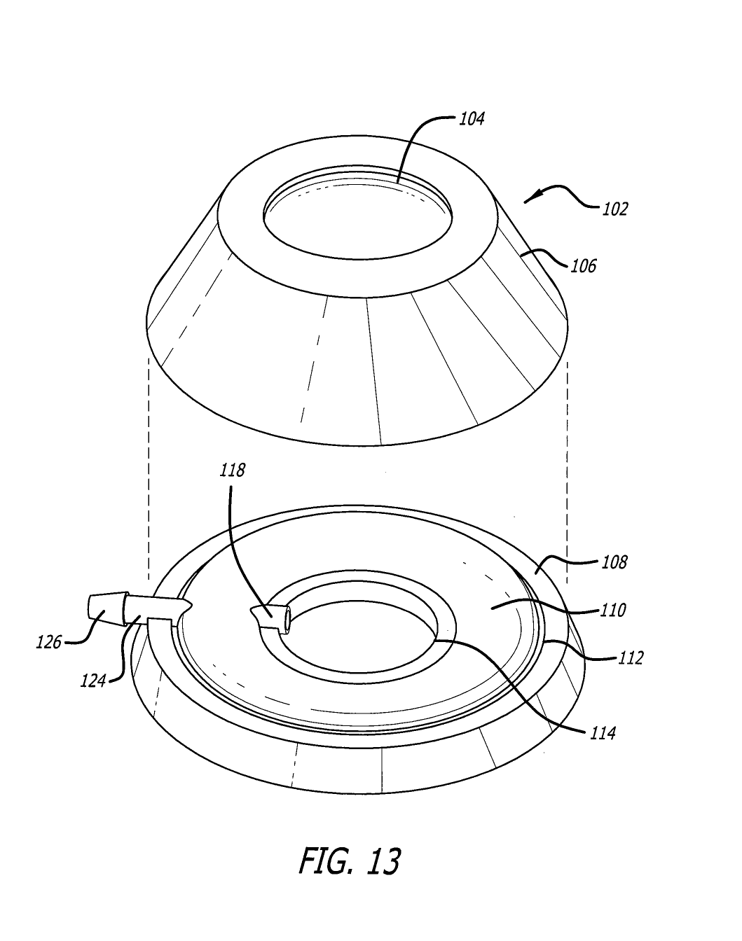

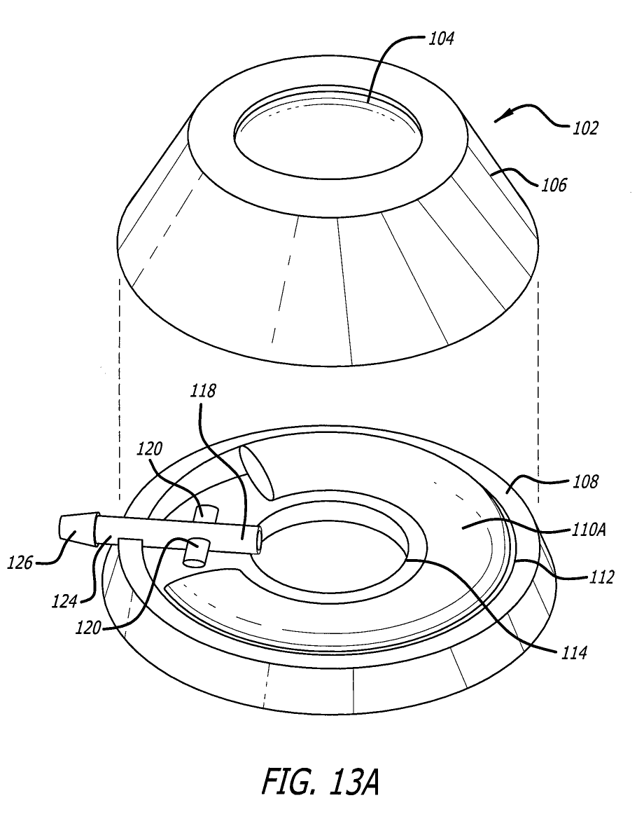

The bladder is mounted in the cavity 108 along a toroidal surface 112 (or within a toroidal chamber or volume). Bladder 110 is shown in FIG. 11 in a deflated configuration and in FIG. 13 in an inflated configuration. Fluid flows into bladder 110 via fluid chamber 114. A cross connector 116 is attached to the bottom cavity 108 and has four arms. First arm 118 extends into fluid chamber 114 and provides a flow pathway from the fluid chamber into the second arm 120 and the third arm 122. Bladder 110 is connected to the second arm 120 and third arm 122 so that fluid from the fluid chamber 114 flows through first arm 118 and second arm 120 and third arm 122 in order to allow fluid flow into and out of bladder 110. A fourth arm 124 is in fluid communication with the first arm 118, second arm 120, and third arm 122. Fluid flows from the fourth arm 124 through tubing (not shown) to the gastric band and into the balloon portion of the gastric band. The fourth arm 124 has a barbed fitting so that the tubing can be securely attached to the fourth arm.

Still with reference to FIGS. 9-13, the injection port bladder assembly 100 is attached to any conventional gastric banding system such as the one shown in FIG. 1. In this embodiment, the port 26 and tubing 24 shown in FIG. 1 is unnecessary, since the injection port bladder assembly 100 replaces the port 26. In further keeping with the invention, the injection port bladder assembly is attached to a gastric band and a conventional syringe is used to inject fluid through septum 104 in order to fill fluid chamber 114. As fluid flows into the fluid chamber, the fluid flows through the cross-connector 116 and fills bladder 110 so that it expands against the toroidal surface 112. Expansion of the bladder is limited against the constraint of the wall of the toroid surface 112 (see FIG. 13). As fluid flows into bladder 110, fluid also flows through cross-connector 116, including through fourth arm 124 and tubing (now shown) to the gastric band, and more particularly into the balloon portion of the gastric band. As set forth above, the bladder 110 and the balloon portion 22 of the gastric band 20 automatically and continuously equalize pressure in the system in response to changes in the restriction surrounded by the balloon portion of the gastric band. Alternatively, as shown in FIG. 13A, the injection port bladder assembly 100 is similar to that shown in FIGS. 9-13. In this embodiment, fluid does not flow into bladder 110a, rather the bladder 110a is filled with a compressible material such as air, foam, micro-bubbles, or a similar compressible material. The bladder 110a is a closed system and prior to injecting fluid into septum 104, the bladder 110a is in an expanded configuration. As fluid is injected into or through septum 104, the fluid fills chamber 114 and flows through first arm 118 and second arms 120 so that the fluid flows around bladder 110a. As the fluid is further injected into the injection port, the fluid compresses bladder 110a which causes the pressure on the fluid to build up so that the pressure on the fluid will flow through fourth arm 124 to the balloon portion of the gastric band. Since the fluid pressure in the injection port bladder assembly 100 is higher than that in the balloon portion of the gastric band, the pressure will automatically and continuously equalize in the system in response to changes in the restriction surrounded by the balloon portion of the gastric band.

Some patients receiving prior art gastric bands may exhibit periods of non-responsiveness so that their weight loss might be sporadic, or in some cases, the patient stops losing weight altogether. The bladder assemblies disclosed herein are particularly useful for these patients because the bladder can be incorporated into gastric bands that already have been implanted. For example, for patients having a Realize band with an infusion port to replenish fluid in the balloon portion of the band, bladders of the type disclosed in FIGS. 9-13A can easily be incorporated into the system. The patient is given a local anesthetic so that the infusion port may be removed by a minimally invasive incision. Thereafter, injection port bladder assembly 100 is implanted minimally invasively and attached to the Realize band via existing tubing or replacement tubing associated with the bladder assembly 100. After the injection port bladder assembly 100 is attached to the Realize band, fluid is injected into the bladder to pressurize the bladder and fluid will automatically flow into the balloon portion of the band. The minimally invasive incision is closed. Thereafter, bladder assembly 100 operates as discussed for FIGS. 9-13A herein in order to maintain the patient's weight loss in the green zone.

In another embodiment, as shown in FIG. 14, a bladder assembly 130 includes an expandable bellows 132 that can be formed from an expandable material such as silicone rubber or the like. The bellows can be formed of other materials as long as it is expandable or contractible in an accordion fashion. A spring 134, which is optional, is used to generate pressure within the bellows 132. The spring 134 is compressed against a wall of housing 136 and at its other end against the bellows 132, in order to apply a compressive force on the bellows. Housing 136 can be of any material that is biocompatible and protects the bladder assembly 130. Fill tubing 138 is connected to one of bellows 132 for adding or removing fluid to the bellows 132. An infusion tubing 140 is connected to the opposite end of the bellows and is in fluid communication with the gastric band assembly, such as the one shown in FIG. 1. In operation, the bellows 132 is filled with a fluid such as saline which causes the bellows to expand against the compressive force of spring 134. Depending upon the compliance of bellows 132, the spring 134 may not be necessary for a particular system. In this embodiment, the fluid pressure between the bellows and the balloon portion of a gastric band automatically and continuously adjust so that there is no lasting pressure differential between the expandable balloon and the bellows, and in so doing, the pressure in the balloon is maintained even though there are changes in fluid volume in the balloon. Even as the volume of fluid in the balloon portion of the band changes in response to loading changes, the pressure between the bellows and the balloon remains substantially constant and adjusts the amount of fluid in each continuously and automatically in response. This embodiment of the invention, as with the others disclosed herein, eliminate the need for frequent visits to the doctor to have the balloon portion of the gastric band refilled in order to maintain the patient in the green zone.

As shown in FIG. 15, a multi-pressure plateau pressure bladder is disclosed to provide a range of fill volumes that correspond to a range of intra-band pressures. Instead of measuring intra-band pressure to determine how much volume should be put into the balloon portion of a gastric band as typically is done with the prior art devices, this embodiment, as with the others disclosed herein, allow setting intra-band pressure based on the volume of fluid injected into the band. Further, the embodiments of the present invention also provide adjustment of pressure within a predetermined and known range by measuring the volume of fluid injected by the bladder into the balloon portion of the gastric band. This result is achieved without intra-band manometry which is too cumbersome and time-consuming to be widely used. As shown in FIG. 15, a bladder assembly 142 includes a multi-compliant bladder 144 encased in a solid housing 146. The multi-compliant bladder 144 consists of multiple inflatable sections or segments each of which has a different compliance. Thus, as shown in FIG. 15, a first bladder section 148, second bladder section 150, and third bladder section 152 form the multi-compliant bladder 144. The first bladder section has the highest compliance and is the most elastic and as fluid is added to the bladder assembly 142, the first bladder section 148 will expand first. In order to shift the compliance into the higher range of the second bladder section, expansion of the first bladder section 148 must be limited. This can be accomplished by using a rigid, solid housing 146 that will constrain each of the bladder sections as they expand. Thus, as fluid is added to the bladder assembly, the first bladder section 148 will expand until it is limited by solid housing 146, thereby increasing the pressure enough to cause expansion or dilation of second bladder section 150. The solid housing 146 also prevents the first bladder section 148 from rupturing. As fluid continues to flow into the bladder assembly 142, the second bladder section 150 will continue to expand or dilate until it also contacts solid housing 146, whereupon the pressure again will increase so that the third bladder section 152 also will expand.

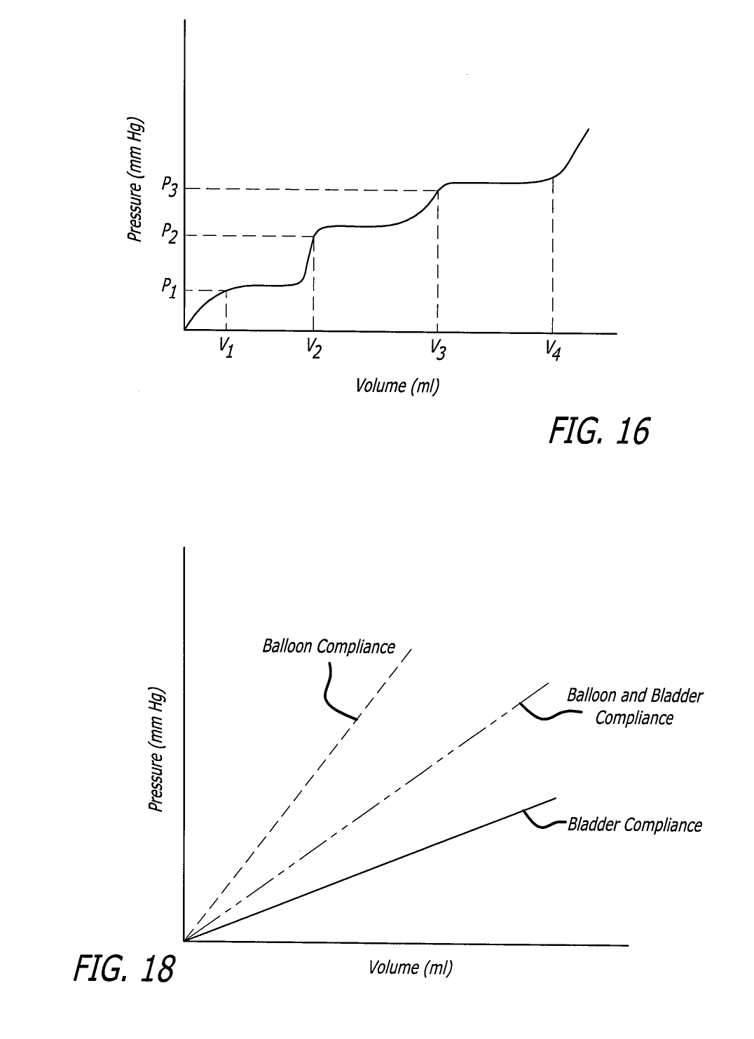

The compliance curves for the embodiment shown in FIG. 15 is shown in FIG. 16. With the use of multi-pressure plateau pressure bladder assembly, a range of fill volumes will correspond to a range of intra-band pressures. Thus, as shown in FIG. 16, for a fill volume between V₁ and V₂, which corresponds to the filling of first bladder section 148, the intra-band pressure (at the balloon's portion of the gastric-band) will be nearly constant at P₁. For a fill volume between V₂ and V₃, which corresponds to the filling of second bladder section 150, the intra-band pressure will be P₂. Likewise, for a volume between V₃ and V₄, the intra-band pressure will be P₃.

In another embodiment, shown in FIGS. 17A-17C, a bladder assembly 160 includes a gastric band 162 and an injection port 164 connected by tubing 166. The tubing 166 is in fluid communication with the gastric band and the balloon portion (not shown) of the gastric band as previously described herein. In this embodiment, some or all of the tubing 166 acts as a bladder. For example, as shown in FIG. 17B, all or a portion of tubing 166 includes a coaxial tubing bladder 168 that extends from the gastric band 162 to the injection port 164. The tubing bladder 168, which is in coaxial alignment with tubing 166, has a first diameter 170 in which there is no fluid flowing through tubing bladder 168. The tubing bladder 168 has a second diameter, that is expanded radially outwardly from fluid being injected into the injection port 164 and flowing into tubing bladder 168. The tubing bladder 168 is formed of an elastic material such as the ones described herein is elastic so that it will expand radially outwardly to second diameter 172. The tubing bladder 168 has a compliance that is lower than the compliance of the balloon portion of the gastric band 162 so that the fluid in tubing bladder 168 is under pressure and will automatically flow into the balloon portion of the gastric band to automatically adjust for patient weight loss as described herein. Similarly, as shown in FIG. 17C, the tubing 166 is separated into two chambers. In this embodiment, bladder 174 is one chamber and it is in fluid communication with the injection port 164 and the balloon portion of the gastric band. The bladder 174 is formed by an outer wall 176 of tubing 166 and a septum 178 that is elastic and is capable of expanding radially outwardly due to fluid pressure within bladder 174. As fluid is injected into injection port 164, the fluid flows into bladder 174 causing the septum 178 to move radially outwardly from it relaxed configuration 180 in the direction of the arrows to its expanded configuration 182. In the expanded configuration, the bladder 174 exerts pressure on the fluid within. The septum 178 is highly elastic and has a lower compliance than the balloon portion of the gastric band, therefore the pressure of the fluid in the bladder 174 will continuously and automatically cause fluid to flow into (or out of) the balloon portion of the gastric band depending upon the changes in the size of the restriction due to the weight gain or the weight loss of the patient.

With respect to the embodiments of the invention disclosed herein, there are a number of different compliance characteristics that may be imparted by the pressure bladder to a gastric banding system. The most appropriate compliance characteristics, both qualitatively and quantitatively, may depend on the compliance characteristics of the gastric band to which the bladder will be made, the desired patient management strategy, and characteristics of the individual patient. Four qualitatively distinct compliance curves are shown in FIGS. 18-21 and described as follows. In FIG. 18, a linearly increasing or decreasing compliance curve is shown, as fluid is injected into the balloon portion of the gastric band, the intra band pressure rises proportionately. Ideally, the slope of the bladder compliance is lower than that of the balloon compliance alone. The addition of the lower slope (higher compliance) bladder to the balloon compliance, increases the compliance of the balloon system. After the bladder has been filled with fluid, then for a given change in balloon fluid volume, there is less of an accompanying change in the intra-band pressure (as compared to the balloon system without the bladder). From a clinical standpoint, in the event of fluid leakage from the balloon, an onset of tissue edema, stoma remodeling, etc., there would be less change to the intra-band pressure. Consequently, the patient may stay in the green zone longer. A linear curve also retains the inherent balloon characteristic of adjustability. Pressure can still be adjusted by adding or removing fluid volume to the system. The slope of the bladder compliance curve has limits. If the balloon system compliance curve is too steep, it will not hold enough fluid volume to meaningfully maintain intra-band pressure. If the bladder system compliance curve is too shallow, it will require too much fluid volume.

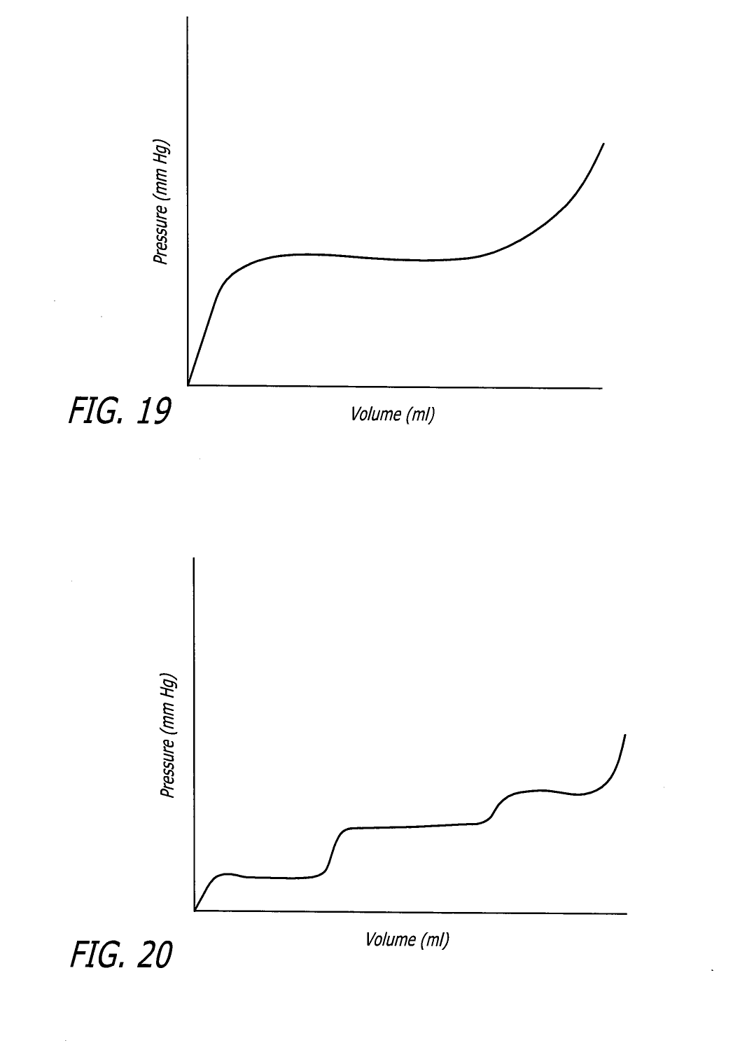

With reference to FIG. 19, a flat or constant pressure compliance curve is shown. In this embodiment, the compliance would keep the intra-band pressure at a substantially constant level over a wide range of volumes. This characteristic may be desirable in maintaining the patient in the green zone without adjustments. In this embodiment, the pressure can be set in a specific range for a specific commercially available gastric band. For example, for the Realize gastric band (Johnson & Johnson) the pressure can be set at 20 mmHg up to 40 mmHg. Similarly, for a Lap-Band AP (Allergan), the pressure range may be set somewhat higher, in the range of 50 mmHg up to 150 mmHg.

Referring to FIG. 20, a multi-staged constant pressure compliance curve is shown. The lack of adjustability of some of the embodiments can be overcome with a multi-plateau compliance curve. In this embodiment, pressure can be based on fill volume. Thus, for any particular fill volume, there will be a corresponding constant pressure until a next level of fill volume is added to the bladder system. The embodiment of the bladder assembly shown in FIG. 15 could produce a compliance curve such as that shown in FIG. 20.

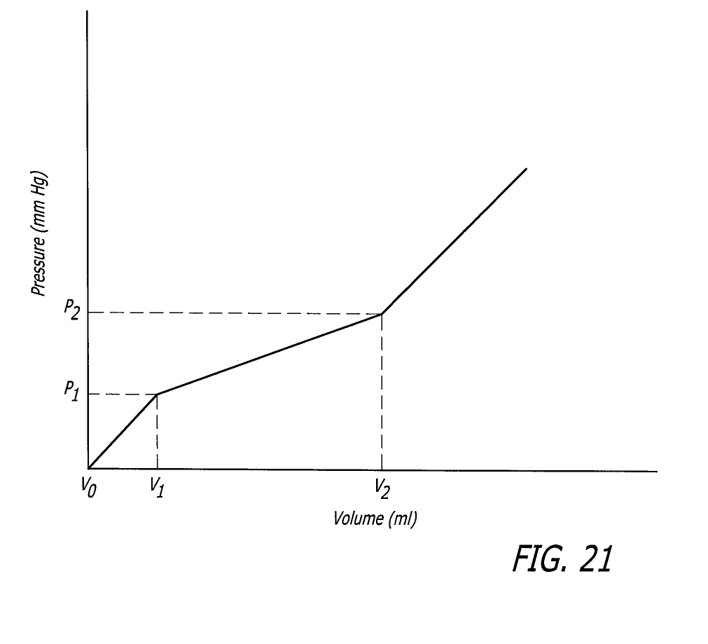

With reference to FIG. 21, a multi-staged linearly increasing compliance curve is shown. In this embodiment, the compliance curves are linearly increasing in staged distinct slopes. In this embodiment, the gastric band would operate between V₁ and V₂. The initial slope, from V₀ to V₁, is steeper in order to reduce the volume of fluid needed to enter the operating zone. The slope in the operating range would be relatively flat, but would allow the surgeon some degree of adjustability. For example, for use with the aforementioned Realize band, the P₁ and P₂ pressures might be 20 mmHg and 40 mmHg respectively.

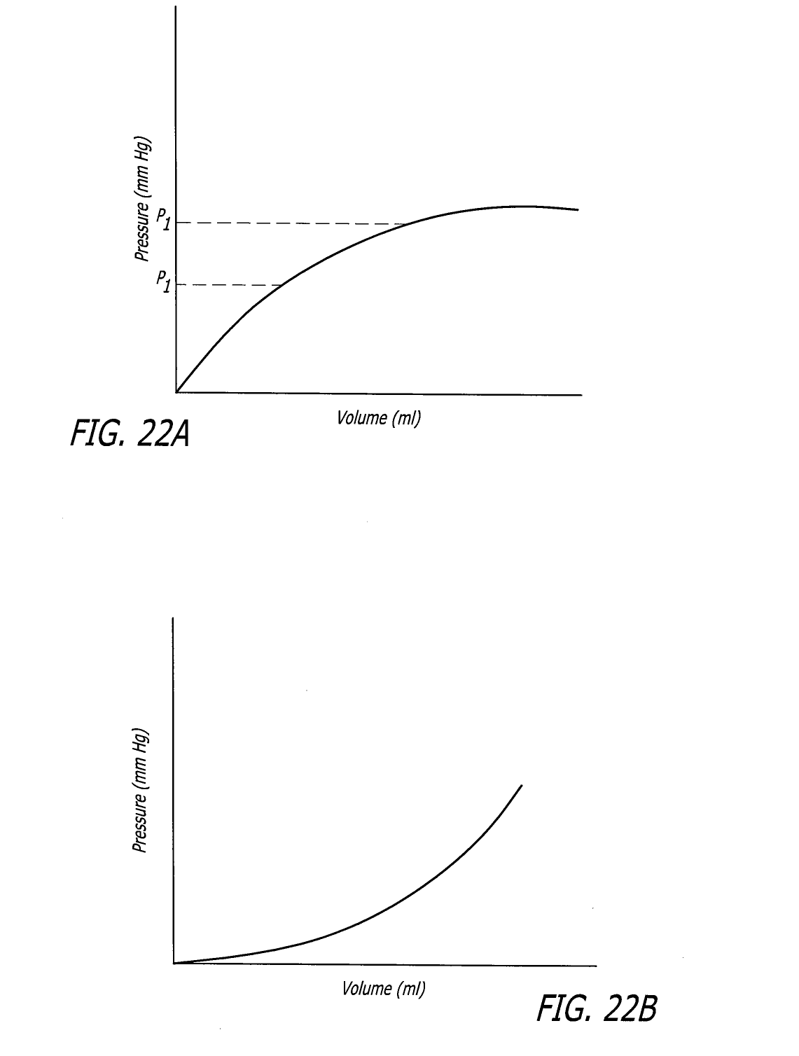

As shown in FIGS. 22A and 22B, exponential and logarithmic compliance curves may be suitable for some patients.

The bladders used with the present invention can be formed from any number of known elastic materials such as silicone rubber, isoprene rubber, latex, or similar materials. As an example, a bladder can be formed by coating silicone rubber on a 0.188 inch outside diameter mandrel to a thickness of about 0.005 inch. Once cured, the silicone rubber coating is removed from the mandrel in the form of a tubing, and can be cut to various lengths in order to form the bladder. As an example, the tubing forming the bladder can range in lengths from 10 mm up to 80 mm, and in one preferred embodiment, is approximately 20-40 mm in length. The tubing can have an outside diameter of approximately 0.125 inch and an inside diameter of 0.0625 inch. The compliance (pressure versus volume) curve of the bladder can vary depending on a number of factors including in the durometer rating of the silicone rubber, the wall thickness of the tubing forming the bladder, and the shape of the bladder.

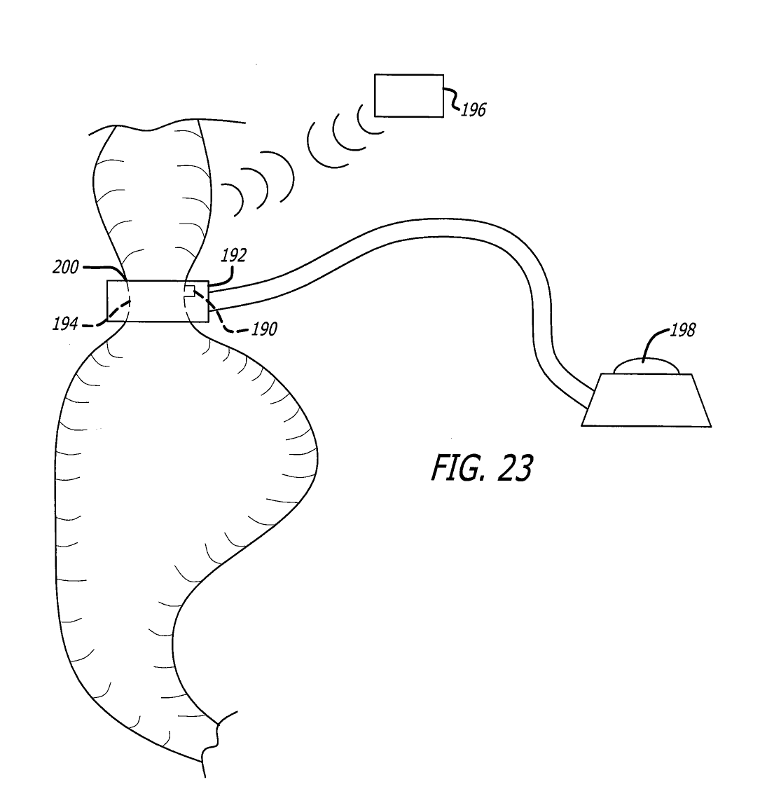

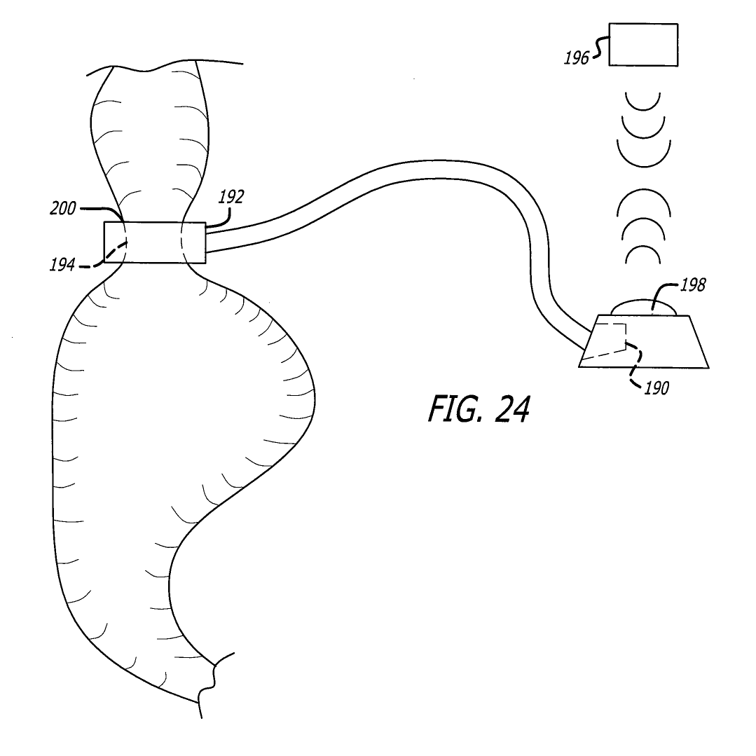

Optionally, the embodiments of the bladder assemblies disclosed herein can incorporate one or more wireless sensors to measure parameters such as pressure, flow, temperature, tissue impedance to detect tissue erosion, slippage of the gastric band, stoma diameter (via ECHO or sonomicrometry) for erosion, slippage or pouch dilatation. These sensors can be implanted in the balloon portion of the gastric band, in the bladder, in the injection port, or anywhere in the system to monitor, for example, pressure. Thus, a sensor could be implanted in the band to measure intra-band pressure or the contact pressure between the gastric band and the tissue enclosed within the band. Similarly, a sensor could be implanted in the bladder to measure fluid pressure within the system. These sensors are wireless and they communicate with an external system by acoustic waves or radio frequency signals (EndoSure® Sensor, CardioMEMS, Inc., Atlanta, Ga. and Ramon Medical Technology, a division of Boston Scientific, Natick, Mass.). In one embodiment, shown in FIG. 23, a pressure sensor 190 is implanted in the gastric band 192 which encircles stoma 194. The sensor 190 communicates a signal wirelessly (using acoustic waves for example) to external system 196 which will analyze the signal. If, as an example, the sensor indicates that the intra-band pressure or the contact pressure between the band and the stoma is low (perhaps 5 mm Hg), this might be an indication that: (1) the bladder 198 has transferred all of its fluid to the balloon portion 200 of band 192 and needs to be refilled; or (2) there is a fluid leak in the system; or (3) the bladder is not working properly to continuously maintain the correct pressure at sensor 190. Alternatively, as shown in FIG. 24, sensor 190 is implanted in injection port bladder assembly 198 to measure fluid pressure. The signal from the sensor 190 is transmitted wirelessly to external system 196 to monitor the pressure in the bladder. If the bladder pressure falls too low, the bladder can be refilled as described above for FIGS. 9-13. By wireless monitoring intra-band pressures, patient management can be improved. For example, if pressures are higher or lower than desired for a given system compliance curve, then fluid can be removed or added respectively to the bladder in the system, after factoring other aspects of the patient's status. If the pressure is in the correct range for a given system, then the surgeon may chose not to adjust the band and instead counsel the patient to improve weight loss by life style improvements.

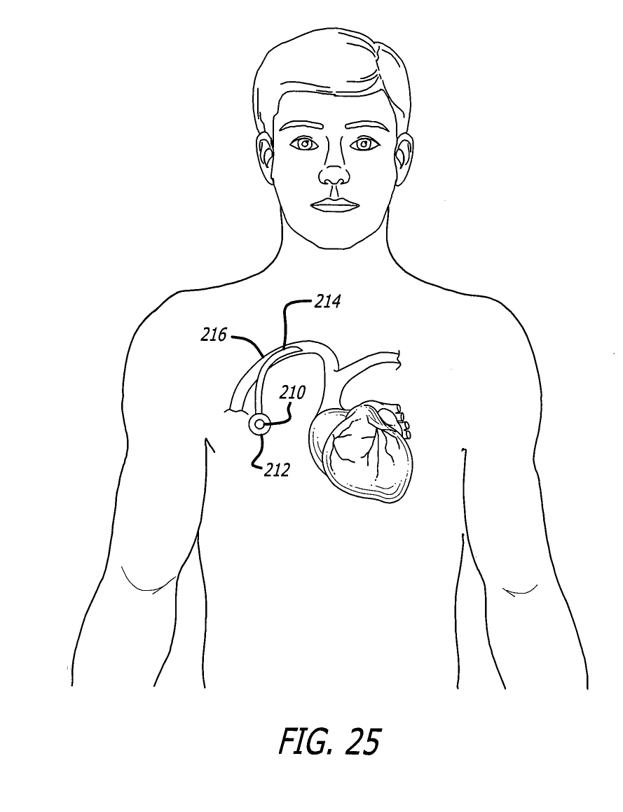

The bladder assembly disclosed herein also can be used with a venous access catheter to reduce the likelihood of clotting or hemostasis in the catheter. One of the greatest challenges with venous access catheters is their propensity to thrombose resulting in a loss of patency. These catheters are typically implanted in the subclavian vein and often include an implanted vascular access port. These vascular access ports and catheters are quite stiff having little or no fluid compliance. Central Venous Pressure is relatively low, ranging normally from 2-6 mm Hg, with a pulsatile waveform. Because of the stiffness of the vascular access ports there is little distension of the inside of the access port in response to the pulsatile venous pressure waveform. Consequently, fluid within the catheter is stagnant. Hemostasis results in coagulation or clot formation. In one embodiment, as shown in FIG. 25, a compliant bladder 210 inside a port 212 may act like a trampoline and distend in response to the pressure waveform. In so doing it may cause the blood or other fluid column inside the catheter 214 to move back and forth constantly. This may prevent or delay hemostasis and clotting and result in a catheter that remains patent longer. In this embodiment, the catheter 214 is inserted in a vessel 216 (vein or artery) for infusion or withdrawal of fluids. Such systems are well known in the art (see e.g., Vital-Port® Vascular Access System, Cook Medical, Bloomington, Ind.).

With respect to any of the embodiments of the bladder disclosed herein, the bladder can be used as a drug delivery reservoir and a drug delivery pump. The bladders have an elasticity that generates a pressure on the fluid in the bladder. A drug can be injected into the bladder so that the bladder fills and expands. Due to the elasticity of the bladder, the fluid/drug is under pressure. The drug can be infused into a patient from the bladder at a controlled rate.

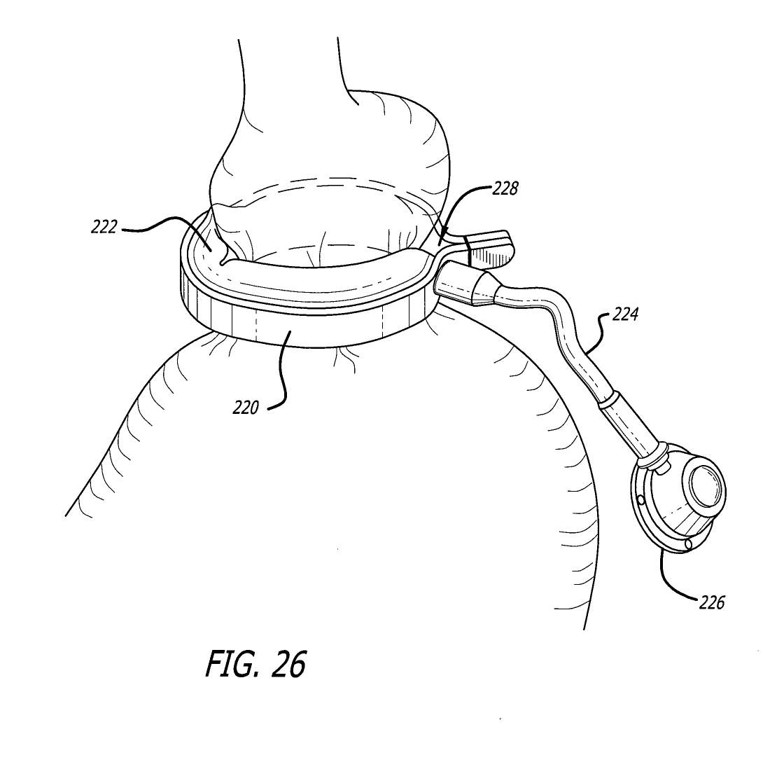

In one alternative embodiment as shown in FIG. 26, the balloon portion 222 of a gastric band 220 is formed of an elastic material so that as the balloon is filled with a fluid, it will elastically expand. In this embodiment, as the stoma encircled by the gastric band 228 gets smaller when the patient loses weight, the balloon portion 222 will expand because fluid from the port 226 and tubing 224 will automatically flow into the balloon in order to keep a constant (predetermined) pressure on the stoma. The port 226 and the tubing 224 contain about 9 ml fluid, so the balloon has a good capacity for expansion as the stoma reduces in size. The port also can be replenished with fluid as described herein.

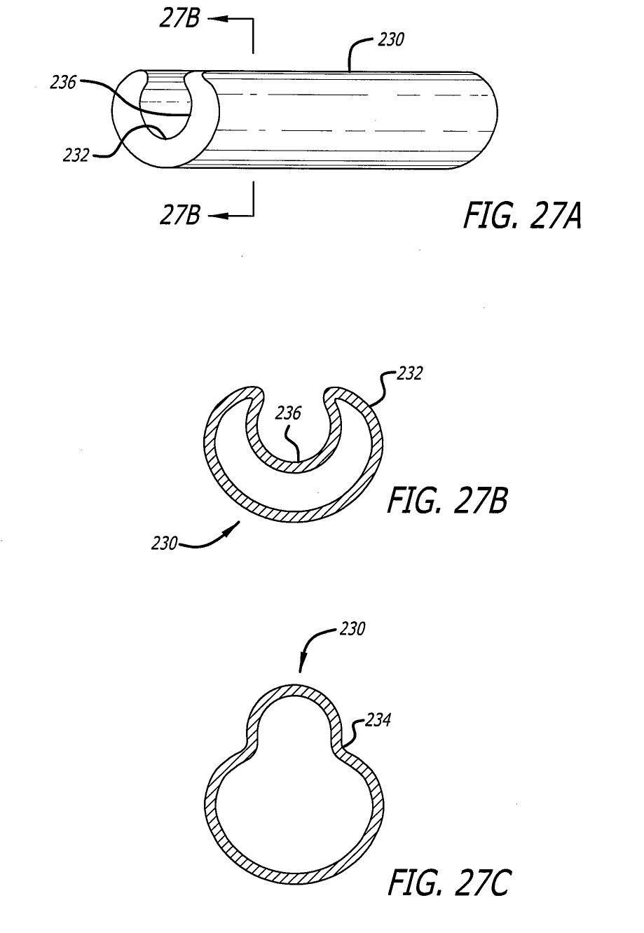

In one embodiment, bladder 230 has a unique cross-sectional shape that will achieve a desired pressure/volume curve utilizing both the material properties of the bladder (elastic material) as well as changing the cross-sectional shape. As shown in FIGS. 27A-27C, the bladder 230 has a folded configuration 232 (FIG. 27B) and an unfolded configuration 234 (FIG. 27C). In the folded configuration 232, the bladder 230 has a longitudinal fold 236 providing a very low profile for minimally invasive delivery. When fluid is then added to the bladder 230, it will pop open or unfold to the unfolded configuration 234 where the elastic properties of the bladder and its unique shape will pressurize the fluid. This embodiment can be incorporated into most of the bladder systems disclosed herein (e.g., FIGS. 2-8, 13, 13A, 15 and 23-26). In another embodiment, the bladder 230 can have more than one longitudinal fold, similar to longitudinal fold 236, spaced around the circumference of the bladder. In the folded configuration, such a bladder would have very low profile for minimally invasive delivery.

1-28. (canceled)

29. A method for maintaining a basal intra-band pressure with a gastric band, comprising:
providing a gastric band assembly having a gastric band which includes a balloon encircling stomach tissue to form a stoma and generating an intra-luminal pressure within the stoma with the gastric band;
the balloon having a pressure-volume compliance curve when filled with a fluid;
incorporating a bladder into the gastric band assembly so that the bladder and the balloon are in fluid communication; and
after incorporating the bladder, the balloon and bladder combination having a pressure-volume compliance curve having a lower slope than the pressure-volume compliance curve of the balloon alone.

30. The method of claim 29, wherein the bladder lessens changes in the intra-band pressure resulting from adding fluid to or removing fluid from the gastric band.

31. The method of claim 29, wherein fluid flows automatically and autonomously between the balloon and the bladder in response to changes in the size of the stoma encircled by the balloon.

32. The method of claim 29, wherein fluid flows automatically and autonomously from the bladder to the balloon to compensate for fluid loss in the balloon due to leaks.

33. The method of claim 29, wherein fluid flows automatically and autonomously from the bladder to the balloon to compensate for a loosening of the band.

34. The method of claim 29, wherein fluid flows automatically and autonomously from the balloon to the bladder to compensate for tightening of the band.

35. The method of claim 29, wherein fluid flows from the balloon to the bladder when the patient swallows, and fluid flows back from the bladder to the balloon after the patient swallows.

36. The method of claim 35, wherein fluid flows from the balloon to the bladder when the patient swallows, and fluid flows automatically and autonomously back from the bladder to the balloon after the patient swallows.

37. The method of claim 29, wherein the balloon has a first compliance before the bladder is incorporated into the gastric band assembly and a second compliance after the bladder is incorporated into the gastric band assembly, the second compliance being greater than the first compliance.

38. The method of claim 29, wherein fluid flows to and from the balloon and the bladder in order to maintain an intra-luminal pressure range from 20 mmHg to 40 mmHg in response to fluid being added to or withdrawn from the gastric band assembly.

39. The method of claim 29, wherein fluid flows to and from the balloon and the bladder in order to maintain an intra-luminal pressure in the range from 20 mmHg to 40 mmHg in response to changes in stoma diameter.

40. A method for maintaining a basal intra-band pressure with a gastric band, comprising:
providing a bladder in fluid communication with a balloon portion of a gastric band; and
maintaining a range of an intra-band pressure from about 10 mmHg to about 30 mmHg in response to the addition to or removal from the gastric band of up to 5 mL fluid volume.

41. The method of claim 40, wherein a pressure-volume curve has a slope of 4 mmHg/1 mL.

42. The method of claim 40, wherein fluid flows automatically and autonomously between the balloon and the bladder in response to changes in the size of the stoma encircled by the balloon.

43. The method of claim 40, wherein fluid flows automatically and autonomously from the bladder to the balloon to compensate for fluid loss in the balloon due to leaks.

44. The method of claim 40, wherein fluid flows automatically and autonomously from the bladder to the balloon to compensate for a loosening of the band.

45. The method of claim 40, wherein fluid flows automatically and autonomously from the balloon to the bladder to compensate for a loosening of the band.

46. The method of claim 40, wherein fluid flows automatically and autonomously between the bladder and the balloon in response to changes in intra-luminal pressure.

47. A method for maintaining a contact pressure on a stoma with a gastric band, comprising:
providing a gastric band assembly having a gastric band and a balloon encircling stomach tissue to form a stoma and generating a contact pressure between the stoma and the gastric band;
incorporating a bladder into the gastric band assembly so that the bladder and the balloon are in fluid communication;
adding or removing fluid within the gastric band assembly to adjust the contact pressure; and
minimizing changes from the adjusted contact pressure as fluid automatically and autonomously flows between the bladder and the balloon.

48. A method for maintaining a level of a pre-set contact pressure on a stoma within a gastric, comprising:
providing a gastric band assembly having a gastric band and a balloon, the balloon encircling the stoma;
incorporating a bladder in the gastric band assembly so that the bladder is in fluid communication with the balloon;
adding or removing fluid to the gastric band to thereby set the contact pressure on the stoma by the gastric band; and
as contact pressure on the stoma changes, fluid flows between the bladder and the balloon to substantially lessen the change in the set contact pressure.

49. The method of claim 48, wherein as a diameter of the stoma decreases, fluid flows automatically and autonomously from the bladder to the balloon to increase the volume of fluid in the balloon and substantially lessen changes to the set contact pressure.

50. The method of claim 48, wherein as a diameter of the stoma increases, fluid flows automatically and autonomously from the balloon to the bladder to decrease the volume of fluid in the balloon and substantially lessen changes to the set contact pressure.

51. The method of claim 48, wherein a generally 20% decrease in stoma diameter generates a generally 7% decrease in intra-band pressure.

52. A method of preventing tightening of a gastric band due to food being stuck above or within the stoma created by the gastric band, comprising:
providing a gastric band assembly having a gastric band and a balloon, the balloon encircling stomach tissue to form a stoma and generating a desired contact pressure at the interface between the stoma and the balloon;
incorporating a bladder in the gastric band assembly and in fluid communication with the balloon; and
preventing tightening of the gastric band due to food being stuck above the gastric band by fluid flowing automatically and autonomously from the balloon to the bladder thereby reducing the volume of fluid in the balloon and increasing a diameter of the stoma so that the food can pass through the stoma and maintain the desired contact pressure.

53. A method of treating a patient having a gastric band, comprising:
providing a gastric band assembly having a balloon, the balloon encircling stomach tissue to form a stoma;
incorporating a bladder in the gastric band assembly, the bladder and the balloon being in fluid communication; and
fluid flows automatically and autonomously between the balloon and the bladder in response to changes in the size of the stoma.

54. A method of treating a patient having a gastric band, comprising:
providing a gastric band assembly having a balloon, the balloon encircling stomach tissue to form a stoma and generating a contact pressure between the stoma and the balloon;
incorporating a bladder in the gastric band assembly, the bladder and the balloon being in fluid communication; and
fluid flows automatically and autonomously between the balloon and the bladder in response to changes in the contact pressure.

55. A method of treating a patient having a gastric band, comprising:
providing a gastric band assembly having a balloon, the balloon encircling stomach tissue to form a stoma;
incorporating a bladder in the gastric band assembly, the bladder and the balloon being in fluid communication; and
fluid flows automatically and autonomously between the balloon and the bladder in response to changes in gastric band tightness.
